(19) United States
(12) Patent Application Publication
(10) Pub. No.: US 2025/0052738 A1
(43) Pub. Date: Feb. 13, 2025

(54) BIOMARKER

(71) Applicants: UNIVERSITY OF LEICESTER, Leicester, Leicestershire (GB); LOUGHBOROUGH UNIVERSITY, Loughborough, Leicestershire (GB)

(72) Inventors: Christopher BRIGHTLING, Leicester (GB); Salman SIDDIQUI, London (GB); Rebecca Lynne CORDELL, Leicester (GB); Michael John WILDE, Plymouth (GB)

(21) Appl. No.: 18/290,718

(22) PCT Filed: Jul. 19, 2022
(86) PCT No.: PCT/GB2022/051858

§ 371 Date: Jan. 19, 2024

(30) Foreign Application Priority Data
Jul. 19, 2021 (GB) 2110365.0

(51) Int. Cl. G01N 30/02; G01N 30/72

(52) U.S. Cl. CPC G01N 33/4975; G01N 30/7206; G01N 2030/025; G01N 2800/122; G01N 2800/325

(57) ABSTRACT

The invention relates to a method of diagnosing and a method of treating a cardiorespiratory disease in a subject.

### FIELD OF THE INVENTION

The invention relates to a method of diagnosing and a method of treating a cardiorespiratory disease in a subject experiencing breathlessness.

### BACKGROUND

Breathlessness due to cardio-respiratory diseases accounts for more than 1 in 8 of all emergency admissions to hospital. Despite the same presenting symptom, the aetiology of acute breathlessness is highly varied, with diverse disease trajectories and treatment options. Diagnostic evaluation of acute breathlessness is heavily reliant on investigations such as blood-based biomarkers (e.g. C-reactive protein (CRP), B-type natriuretic peptide (NT-pro BNP)) and radiological procedures. These biomarkers have clinical utility primarily in patients with single pathologies, but have poor discriminatory power in patients with multifactorial presentations of acute breathlessness and are particularly challenging to interpret in the context of pre-admission treatment exposure (e.g. antibiotics for pneumonia and admission CRP values). Additionally, delays in blood sample processing at the point of triage can result in inappropriate treatment decisions and consequently harmful effects to patients. To address these issues, there have been considerable advancements in the field of metabolomics, underpinned by analytical technologies, which permit comprehensive identification and quantification of metabolite profiles in biological systems from samples acquired at the point of clinical care.

Nevertheless, there is a need for biomarkers that can be used to diagnose and distinguish between cardiorespiratory conditions that present with breathlessness as a symptom.

### STATEMENTS OF INVENTION

According to a first aspect of the invention, there is provided a method of diagnosing a cardiorespiratory disease in a subject, the method comprising:
detecting the presence of one or more cardiorespiratory disease-VOC biomarkers in a sample of exhaled breath from the subject,
wherein if one or more of the VOC biomarkers is present in the sample, the subject may have a cardiorespiratory disease.

In one embodiment, there is provided a method of diagnosing asthma in a subject, the method comprising:
detecting the presence of one or more asthma-VOC biomarkers in a sample of exhaled air from the subject,
wherein if one or more of the VOC biomarkers is present in the sample, the subject may have asthma.

In one embodiment, there is provided a method of diagnosing COPD in a subject, the method comprising:
detecting the presence of one or more COPD-VOC biomarkers in a sample of exhaled air from the subject,
wherein if one or more of the VOC biomarkers is present in the sample, the subject may have COPD.

In one embodiment, there is provided a method of diagnosing pneumonia in a subject, the method comprising:
detecting the presence of one or more pneumonia-VOC biomarkers in a sample of exhaled air from the subject,
wherein if one or more of the VOC biomarkers is present in the sample, the subject may have pneumonia.

In one embodiment, there is provided a method of diagnosing heart failure in a subject, the method comprising:
detecting the presence of one or more heart failure-VOC biomarkers in a sample of exhaled air from the subject,
wherein if one or more of the VOC biomarkers is present in the sample, the subject may have heart failure.

According to a second aspect, there is provided a method of treating a cardiorespiratory disease in a subject, the method comprising:
detecting the presence of one or more cardiorespiratory disease-VOC biomarkers in a sample of exhaled air from the subject,
wherein the presence of one or more of the VOC biomarkers in the sample suggests the subject has a cardiorespiratory disease, and
administering a therapeutic agent to the subject, in order to treat the cardiorespiratory disease.

According to a third aspect, there is provided a method of treating a cardiorespiratory disease in a subject, the method comprising:
administering a therapeutic agent to the subject, who has been diagnosed with a cardiorespiratory disease using the method according to the invention.

According to fourth aspect, there is provided a method of selecting a subject for treatment with a therapeutic agent or composition for a cardiorespiratory disease, the method comprising:
detecting the presence of one or more cardiorespiratory disease-VOC biomarkers in a sample of exhaled air from the subject,
wherein the presence of one or more of the VOC biomarkers in the sample suggests the subject has a cardiorespiratory disease, and
selecting the subject for treatment with a therapeutic agent or composition for the cardiorespiratory disease.

According to another aspect, there is provided a method of selecting a subject for treatment with a therapeutic agent or composition for a cardiorespiratory disease, the method comprising:
selecting a subject, who has been diagnosed with a cardiorespiratory disease using the method according to the invention, for treatment with a therapeutic agent or composition for a cardiorespiratory disease.

The invention provides a more patient-compliant method of diagnosing and treating a cardiorespiratory disorder. The invention enables a subject to be diagnosed without the use of invasive procedures, such as taking blood, or radiological processes. The method may not be performed on the subject.

Two important features of any biomarker that are used for diagnostic purposes are sensitivity and specificity. The higher the degree of sensitivity, the lower the probability of generating a false negative. The higher the degree of specificity, the lower the probability of generating a false positive. The biomarkers disclosed herein can surprisingly exhibit up to 79% sensitivity and 85% specificity (with an AUC of 0.89) when distinguishing between individuals with a cardiorespiratory disease and healthy individuals (controls).

The values for differentiating each acute cardiorespiratory disease group from the other acute cardiorespiratory disease groups (i.e. not against healthy patients) are as follows:
Asthma-sensitivity 0.75 (0.63, 0.85), specificity 0.90 (0.85, 0.94);
COPD-sensitivity 0.66 (0.52, 0.78), specificity 0.89 (0.85, 0.93):
Heart failure-sensitivity 0.64 (0.48, 0.78), specificity 0.96 (0.92, 0.98); and.
Pneumonia-sensitivity 0.65 (0.51, 0.78), specificity 0.93 (0.89, 0.96).

The invention thus enables a clinician to make a more informed decision about the diagnosis and treatment of a subject experiencing breathlessness and suffering from a cardiorespiratory disorder.

According to a fifth aspect, there is provided a method of determining if a therapeutic agent or composition is effectively treating a cardiorespiratory disease in a subject, the method comprising:
determining the concentration of one or more cardiorespiratory disease-VOC biomarkers in a test sample that has been exhaled by the subject, and
comparing the concentration of the at least one or more VOCs in the test sample with the concentration in a reference sample,
wherein if the concentration of the one or more VOC biomarkers in the test sample is lower compared to the concentration in a reference sample, it is indicative that the therapeutic agent or composition is effectively treating the cardiorespiratory disease in the subject.

It will be appreciated that the concentration of a VOC biomarker in a test sample positively correlates with the magnitude/severity of the cardiorespiratory disease.

Thus, for example, a reduction in concentration of a VOC biomarker in the test sample compared to the concentration in a reference sample may be indicative of a reduction in the magnitude/severity of the cardiorespiratory disease. Similarly, an increase in concentration of a VOC biomarker in the test sample compared to the concentration in a reference sample may be indicative of an increase in the magnitude/severity of the cardiorespiratory disease.

The concentration of the VOC biomarker in the test sample may be lower by (or reduced by at) least about 5%, 10%, 15%, 20%, 25%, 30%, 35%, 40%, 45%, 50%, 55%, 60%, 65%, 70%, 75%, 80%, 85%, 90%, 95% or 100% compared to the concentration in the reference sample.

The reference sample may have been taken from the same subject or a different subject. Preferably, the reference sample is a sample that has been taken from the same subject but at an earlier time point than the test sample. Preferably the earlier sample indicates that the subject has a cardiorespiratory disease.

Preferably the subject referred to herein is experiencing breathlessness. Preferably, a two-dimensional gas chromatography coupled with mass spectrometry is used to detect the presence of one or more VOC biomarkers in the sample.

A cardiorespiratory disease may be a disease or disorder of the cardiovascular system and/or a disease of the respiratory system. Examples of cardiorespiratory diseases include asthma, COPD, heart failure and a respiratory infection (e.g. pneumonia), bronchitis, emphysema, congestive heart failure, hypertension, angina, peripheral vascular disease and myocardial infarction. Preferably, the term “cardiorespiratory disease” refers to one or more diseases selected from the group comprising: asthma, COPD, heart failure and pneumonia.

A VOC (volatile organic compound) may be referred to as an organic compound that has a boiling point between about 50° C. and about 250° C. at a standard atmospheric pressure of 101.3 kPa.

A cardiorespiratory disease-VOC biomarker may be one or more selected from the group comprising: hydrocarbons, ketones, aldehydes, alcohols, oxygen-containing VOCs, terpenoids, aromatics, sulphur-containing VOCs, nitrogen-containing VOCs, a halogenate (e.g. dichloromethane) and surfactants and emollients.

It will be appreciated that the step of detecting the presence of one or more cardiorespiratory disease-VOC biomarkers may comprise using the method according to the invention. It will also be appreciated that the detection of one more cardiorespiratory disease-VOC biomarkers in a sample is indicative that the subject (from which the sample has been taken) has a cardiovascular disease.

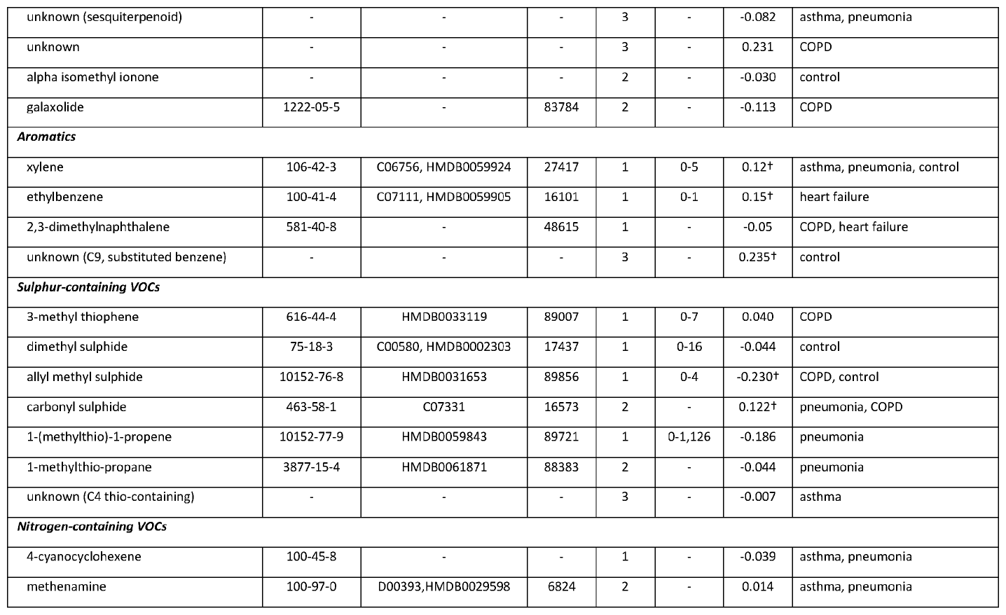

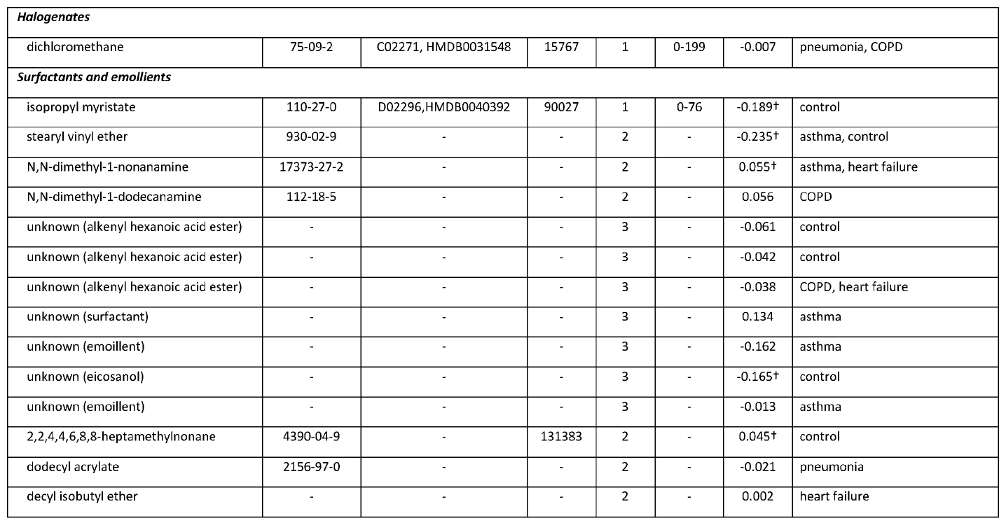

The hydrocarbon VOC may be one or more selected from the group comprising: 2-methylbutane; isoprene: 3-methylpentane: 2,4-dimethylpentane: 2,2-dimethylpentane; hexane: octane: 2,6-dimethyloctane: nonane; 2-methylnonane: 5-methylnonane: decane: 4-methyldecane: undecane: 4-methylundecane: dimethylundecane isomer: 3-methyltridecane; tetradecane: octadecane: 1-nonene; 1-decene; cyclohexane; a cyclohexadiene isomer: methylcyclopentadiene: and a hexadecene isomer. The hydrocarbon may be one or more selected from FIG. 16. Preferably the hydrocarbon VOC may be one or more selected from the group comprising: hexane: octane: 2,6-dimethyloctane; nonane; 2-methylnonane; decane; undecane; 4-methylundecane; dimethylundecane isomer: 3-methyltridecane; tetradecane; octadecane; 1-nonene: 1-decene; cyclohexane; a cyclohexadiene isomer; methylcyclopentadiene; and a hexadecene isomer. The hydrocarbon may be one or more selected from FIG. 16.

The ketone VOC may be one or more selected from the group comprising: acetone: 2,3-butanedione; 2-pentanone; 3-buten-2-one (methyl vinyl ketone); 4-methyl-2-pentanone; 6-methyl-5-hepten-2-one; and cyclohexanone. The ketone may be one or more selected from FIG. 16. Preferably the ketone VOC may be one or more selected from the group comprising 3-buten-2-one (methyl vinyl ketone); 4-methyl-2-pentanone; and 6-methyl-5-hepten-2-one.

The aldehyde VOC may be one or more selected from the group comprising: butanal: hexanal; nonanal; decanal; methyldecanal isomer; undecanal; 2-methyl-2-propenal (methacrolein); 3-methylbenzaldehyde; and tridecanal. The aldehyde may be one or more selected from FIG. 16. Preferably the aldehyde VOC is one or more selected from the group comprising butanal; methyldecanal isomer; undecanal: 2-methyl-2-propenal (methacrolein); 3-methylbenzaldehyde; and tridecanal.

The alcohol VOC may be one or more selected from the group comprising 2-propanol; 2-ethylhexanol; 1-decanol; and 1-hexadecanol. The alcohol may be one or more selected from FIG. 16.

The oxygen-containing VOCs may be one or more selected from the group comprising: ethyl acetate: tetrahydrofuran; 1,4-dioxane; 2-methyl-1,3-dioxolane; and 1,3-dioxolane. The oxygen-containing VOC may be one or more selected from FIG. 16.

The terpenoid VOC may be one or more selected from the group comprising: limonene: alpha-pinene; eucalyptol; menthone: menthol: camphene: p-mentha-1,4/8-diene; 3-carene; beta myrcene; beta-phellandrene; geranylacetone: beta-bisabolene; alpha isomethyl ionone; and galaxolide. The terpenoids may be one or more selected from FIG. 16.

The aromatic VOC may be one or more selected from the group comprising: xylene; ethylbenzene; 2,3-dimethylnaphthalene; and a substituted benzene. The aromatic may be one or more selected from FIG. 16.

The sulphur-containing VOC may be one or more selected from the group comprising 3-methyl thiophene; dimethyl sulphide; allyl methyl sulphide: carbonyl sulphide: 1-(methylthio)-1-propene; and 1-methylthio-propane. The sulphur-containing VOC may be one or more selected from FIG. 16. Preferably the sulphur-containing VOC may be one or more selected from the group comprising dimethyl sulphide; 1-(methylthio)-1-propene; and 1-methylthio-propane.

The nitrogen-containing VOC may be one or more selected from the group comprising: 4-cyanocyclohexene; and methenamine. The nitrogen-containing VOC may be one or more selected from FIG. 16.

The surfactant and emollient VOC may be one or more selected from the group comprising: isopropyl myristate; stearyl vinyl ether; N,N-dimethyl-1-nonanamine; N, N-dimethyl-1-dodecanamine; an alkenyl hexanoic acid ester; 2,2,4,4,6,8,8-heptamethylnonane; dodecyl acrylate; and decyl isobutyl ether. The surfactant and emollient may be one or more selected from FIG. 16.

A cardiorespiratory disease-VOC biomarker may be any combination of the VOC biomarkers disclosed in FIG. 16. Thus, a cardiorespiratory disease-VOC biomarker may be one or more selected from FIG. 16. The VOC may be an isomer of a VOC disclosed in FIG. 16. Thus, a cardiorespiratory disease-VOC biomarker may be one or more VOC biomarkers selected from FIG. 16, or an isomer thereof. An isomer may be a structural isomer, a diastereomer (e.g. cis-trans isomer or a rotamer) or an enatiomer.

In one embodiment, a selection of one or more (e.g. all) of the following VOC biomarkers is used to diagnose a cardiorespiratory disease in a subject: hexane; octane; tetradecane,2,3-butanedione; hexanal: 2-methyl-2-propenal; 1-hexadecanol: 2-methyl-1,3-dioxolane; limonene; eucalyptol; menthone; p-mentha-1,4/8-diene: 3-carene; beta phellandrene; sesquiterpenoid; xylene: 2,3-dimethylnapthalene; carbonyl sulphide: 4-cyanocylohexene; methenamine; dichloromethane; N, N-dimethyl-1-nonanamine; and a alkenyl hexanoic acid ester.

In another embodiment, a selection of one or more (e.g. all) of the following VOC biomarkers is used to diagnose asthma in a subject: 3-methylpentane: hexane; 2-methylnonane; decane: tetradecane; 1-nonene; 2,3-butanedione; 2-pentanone; hexanal; nonanal; decanal; methyldecanal isomer; undecanal: 3-methylbenzaldehyde: 2-ethylhexanol; 1-hexadecanol; tetrahydrofuran; 1,4-dioxane; 2-methyl-1,3-dioxolane; eucalyptol; p-mentha-1,4/8-diene; 3-carene; beta-phellandrene; beta-bisabolene; sesquiterpenodf; xylene: 4-cyanocyclohexene; methenamine: stearyl vinyl ether; N,N-dimethyl-1-nonanamine; and N, N-dimethyl-1-dodecanamine.

A selection of one or more (e.g. all) of the following VOC biomarkers may be used to diagnose asthma in a subject: 3-methylpentane; hexane: 2-methylnonane; decane; tetradecane; 1-nonene; 2,3-butanedione; methyldecanal isomer; undecanal: 3-methylbenzaldehyde; 2-ethylhexanol; 1-hexadecanol; tetrahydrofuran: 1,4-dioxane; 2-methyl-1,3-dioxolane; eucalyptol; p-mentha-1,4/8-diene; 3-carene; beta-phellandrene; beta-bisabolene; sesquiterpenoid; xylene; 4-cyanocyclohexene; methenamine; stearyl vinyl ether: N, N-dimethyl-1-nonanamine: and N, N-dimethyl-1-dodecanamine.

Preferably a selection of one or more (e.g. all) of the following VOC biomarkers is used to diagnose asthma in a subject: 3-methylpentane; 2-methylnonane; decane; 1-nonene; 2-pentanone; nonanal; decanal; methyldecanal isomer; undecanal; 3-methylbenzaldehyde; 2-ethylhexanol; tetrahydrofuran; 1,4-dioxane; beta-bisabolene; and N,N-dimethyl-1-dodecanamine.

Most preferably a selection of one or more (e.g. all) of the following VOC biomarkers is used to diagnose asthma in a subject; 3-methylpentane; 2-methylnonane; decane; 1-nonene; methyldecanal isomer; undecanal; 3-methylbenzaldehyde; 2-ethylhexanol; tetrahydrofuran; 1,4-dioxane; beta-bisabolene; and N, N-dimethyl-1-dodecanamine.

In another embodiment, a selection of one or more (e.g. all) of the following VOC biomarkers is used to diagnose COPD in a subject: octane; nonane; 4-methylundecane; cyclohexane; methylcyclopentadiene; 2,3-butanedione; 6-methyl-5-hepten-2-one; 1-decanol; eucalyptol; 2-methyl-1,3-dioxolane; limonene; menthol; camphene; menthone; galaxolide; 2,3-dimethylnapthalene; carbonyl sulphide; 3-methyl thiophene; alkenyl hexanoic acid ester; allyl methyl sulphide; dichloromethane; and N, N-dimethyl-1-dodecanamine.

A selection of one or more (e.g. all) of the following VOC biomarkers may be used to diagnose COPD in a subject: octane; nonane; 4-methylundecane; cyclohexane; methylcyclopentadiene; 6-methyl-5-hepten-2-one; 1-decanol; eucalyptol; 2-methyl-1,3-dioxolane; limonene; menthol; camphene; menthone; galaxolide: 2,3-dimethylnapthalene; 3-methyl thiophene; alkenyl hexanoic acid ester; dichloromethane; and N, N-dimethyl-1-dodecanamine.

Preferably a selection of one or more (e.g. all) of the following VOC biomarkers is used to diagnose COPD in a subject: nonane: 4-methylundecane; 1-decanol; menthol; camphene; galaxolide; 3-methyl thiophene; and N, N-dimethyl-1-dodecanamine.

In another embodiment, a selection of one or more (e.g. all) of the following VOC biomarkers is used to diagnose heart failure in a subject: isoprene; hexane; 5-methylnonane; 4-methyldecane; undecane; cyclohexene; acetone; butanal; 2-methyl-2-propenal; tridecanal; ethyl acetate; 1,3-dioxolane; limonene; 3-carene; beta myrcene; ethylbenzene; 2, 3-dimethylnapthalene; N, N-dimethyl-1-nonanamine; 2-methyl-2-propenal (methacrolein); alkenyl hexanoic acid ester; and decyl isobutyl ether.

A selection of one or more (e.g. all) of the following VOC biomarkers may be used to diagnose heart failure in a subject: hexane; undecane; cyclohexene; acetone: butanal; 2-methyl-2-propenal; tridecanal: ethyl acetate; 1,3-dioxolane; limonene: 3-carene; beta myrcene; ethylbenzene: 2,3-dimethylnapthalene; N, N-dimethyl-1-nonanamine; 2-methyl-2-propenal (methacrolein); alkenyl hexanoic acid ester; and decyl isobutyl ether.

Preferably a selection of one or more (e.g. all) of the following VOC biomarkers is used to diagnose heart failure in a subject: isoprene; 5-methylnonane; 4-methyldecane; undecane; cyclohexene; butanal; 2-methyl-2-propenal; tridecanal; ethyl acetate; 1,3-dioxolane; beta myrcene; ethylbenzene; and decyl isobutyl ether.

Most preferably a selection of one or more (e.g. all) of the following VOC biomarkers is used to diagnose heart failure in a subject: undecane; cyclohexene; butanal; 2-methyl-2-propenal; tridecanal; ethyl acetate; 1,3-dioxolane; beta myrcene; ethylbenzene; and decyl isobutyl ether.

In another embodiment, a selection of one or more (e.g. all) of the following VOC biomarkers is used to diagnose pneumonia in a subject: 2-methylbutane; 2,4-dimethylpentane; 2,2-dimethylpentane; hexane; octane; 2,6-dimethyloctane; diemthylundecane isomer; tetradecane; p-mentha-1,4/8-diene; 1-decene; 3-buten-2-one (methyl vinyl ketone); cyclohexanone; hexanal; 2-methyl-2-propenal; 2-propanol; 1-hexadecanol; alpha-pinene; menthone; beta-phellandrene; sesquiterpenoid; xylene; carbonyl sulphide; 1-(methylthio)-1-propene; 2-methyl-2-propenal (methacrolein); 1-methylthio-propane; 4-cyanocyclohexene; methenamine; dichloromethane; and dodecylacryalte.

A selection of one or more (e.g. all) of the following VOC biomarkers may be used to diagnose pneumonia in a subject: hexane; octane; 2,6-dimethyloctane; diemthylundecane isomer; tetradecane; p-mentha-1,4/8-diene; 1-decene; 3-buten-2-one (methyl vinyl ketone); hexanal; 2-methyl-2-propenal; 2-propanol; 1-hexadecanol; alpha-pinene; menthone; beta-phellandrene; sesquiterpenoid; xylene; carbonyl sulphide; 1-(methylthio)-1-propene; 2-methyl-2-propenal (methacrolein); 1-methylthio-propane; 4-cyanocyclohexene; methenamine; dichloromethane; and dodecylacryalte.

Preferably a selection of one or more (e.g. all) of the following VOC biomarkers is used to diagnose pneumonia in a subject: 2-methylbutane; 2,4-dimethylpentane; 2,2-dimethylpentane; 2,6-dimethyloctane; diemthylundecane isomer; 1-decene; 3-buten-2-one (methyl vinyl ketone); 1-(methylthio)-1-propene; 1-methylthio-propane; and dodecylacryalte.

Most preferably a selection of one or more (e.g. all) of the following VOC biomarkers is used to diagnose pneumonia in a subject: 2,6-dimethyloctane; diemthylundecane isomer; 1-decene; 3-buten-2-one (methyl vinyl ketone); 1-(methylthio)-1-propene; 1-methylthio-propane; and dodecylacryalte.

One or more of the cardiorespiratory disease-VOC biomarkers disclosed herein (e.g. asthma-VOC biomarkers, COPD-VOC biomarkers, pneumonia-VOC biomarkers, or heart failure-VOC biomarkers) may be a selection of one or more of the biomarkers disclosed above for diagnosing a cardiorespiratory disease in a subject.

Preferably, the one or more (e.g. all) of the following VOC biomarkers is used to diagnose a cardiorespiratory disease in a subject experiencing breathlessness.

Detection of a single VOC biomarker may be used to diagnose a cardiorespiratory disease in a subject. However, it will be appreciated that the more VOC biomarkers that are used in the invention, the more reliably a cardiorespiratory disease can be diagnosed in subject. In other word, the more VOC biomarkers that are used in the invention, the higher the sensitivity and the higher the specificity of the invention. Thus, the invention may comprise detecting two or more, three or more, four or more, five or more, six or more, seven or more, eight or more, nine or more, 10 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, or all of the VOC biomarkers disclosed herein (e.g. the biomarkers disclosed in FIG. 16, or the biomarkers specific for each cardiorespiratory disease disclosed herein). Preferably, the invention comprises determining the presence of five or more, or 10 or more VOC biomarkers.

Detecting the presence of a biomarker may comprise detecting the presence, absence, or the level of the biomarkers. Detecting the presence of a biomarker may comprise the detecting of a level of the biomarker. Detecting the presence or level of a biomarker may comprise determining the concentration of the biomarker(s) in the sample.

It will be appreciated that the absence or presence and/or concentration of a VOC may be detected or determined using any suitable method/technique/technology known in the art, such as two-dimensional gas chromatography coupled with mass spectrometry (GCxGC-MS), gas chromatograph-ion mobility spectrometry (GC-IMS) technology, Gas Chromatograph (GC), Gas Chromatograph-Mass Spectrometry (GCMS), Mass Spectrometry (MS), Ion Mobility Spectrometry (IMS), Differential Mobility Spectrometry (DMS), light absorption Spectrometry, Field Asymmetric Ion Mobility Spectrometry (FAIMS), Electronic Nose, Selective-Ion Flow Tube Mass Spectrometry (SIFT-MS), Protein-transfer-reaction-MS, Optical absorbance/Non-dispersive Infra-red and gas sensors (individual or in an array). Preferably, detecting the absence, presence and/or concentration of a VOC in a sample of exhaled breath from a subject comprises two-dimensional gas chromatography coupled with mass spectrometry (GCxGC-MS). Using GCxGC-MS to detect the absence or presence and/or concentration of a VOC provides unparalleled separation of VOC biomarkers with definitive identification of VOC biomarkers.

It will be appreciated that the sample may be analysed immediately after being taken from the subject (i.e. it may be a fresh sample). The sample may be placed in a sealed container, such as a universal or a bijoux. The sample may be stored. Preferably, the sample is stored in a sealed/sealable container, such as a tube, universal or a bijoux. Preferably the container comprises/contains a sorbent material. Thus, the container may be a sealable container (e.g. tube) comprising/containing sorbent material. The sample may be stored for up to 48 hours. The sample may be stored at a temperature between about 2° C. and about 8° C., or a temperature between about 3° C. and about 6° C.

Preferably the sample is stored at a temperature of about 4° C. Thus, the sample may be stored at a temperature between about 2° C. and about 8° C., a temperature between about 2° C. and about 5° C., a temperature between about 3° C. and about 6° C., or at a temperature of about 4° C., for about 48 hours.

The sample may be dry purged in to reduce the water content of the sample to below 2 mg per tube. Dry purging may be performed by purging the sample with nitrogen gas. Preferably, the dry purging (e.g. dry purging using nitrogen gas) is performed within 48 hours of the sample being collected from the subject. “selecting the subject for treatment” may refer to recording the name and/or an identifier of the subject so that a third party is aware that the subject must be treated with a therapeutic agent or composition for a cardiorespiratory disease.

The term “recording” can refer to fixing or storing in writing (e.g. typed) or digitally (e.g. as a video or voice recording, or on a computer).

The subject may be a person suspected of having a cardiorespiratory disease (e.g. asthma, COPD, heart failure and/or pneumonia). Preferably the subject is experiencing breathlessness. The term “breathlessness”, which is also known as dyspnoea, refers to difficulty breathing. This may be in the form of fast shallow breaths, noisy breathing, wheezing, or using your shoulders and/or muscles of your upper chest to help you breathe.

The subject may be a vertebrate, mammal or domestic mammal. Hence, the method according to the invention may be used to diagnose or treat any animal, for example, pigs, cats, dogs, horses, sheep or cows. Preferably, the subject is a human.

Some or all of the steps of the method of the invention may be carried out in vitro, ex vivo or in vivo.

The method according to the invention may comprise providing a sample obtained from a subject. Thus, the term ‘sample of exhaled air breath’ refers to gas and/or liquid exhaled by a subject, preferably gas and/or liquid (condensate) exhaled from the lungs of the subject. The sample is exhaled from the nose and/or mouth of the subject. Preferably, the sample is an exhaled gaseous sample. Thus, the method of the invention may not be performed on the subject. The amount of the sample may be an amount that provides sufficient biomarker to be measured, for example the sample may be of 500 mL to 1 L.

The term ‘treating’ can refer to preventing, eradicating or reducing the severity of a cardiorespiratory disease. Thus, the therapeutic agent or composition referred to herein may be any agent that prevents, eradicates or reduce the severity of asthma, COPD, heart failure or pneumonia.

The term “comprising” may refer to “consisting of” or “consisting essentially of”.

All of the embodiments and features described herein (including any accompanying claims, abstract and drawings), and/or all of the steps of any method or process so disclosed, may be combined with any of the above aspects or embodiments in any combination, unless stated otherwise with reference to a specific combinations, for example, combinations where at least some of such features and/or steps are mutually exclusive.

For a better understanding of the invention, and to show embodiments of the invention may be put into effect, reference will now be made, by way of example, to the accompanying drawings, in which:—

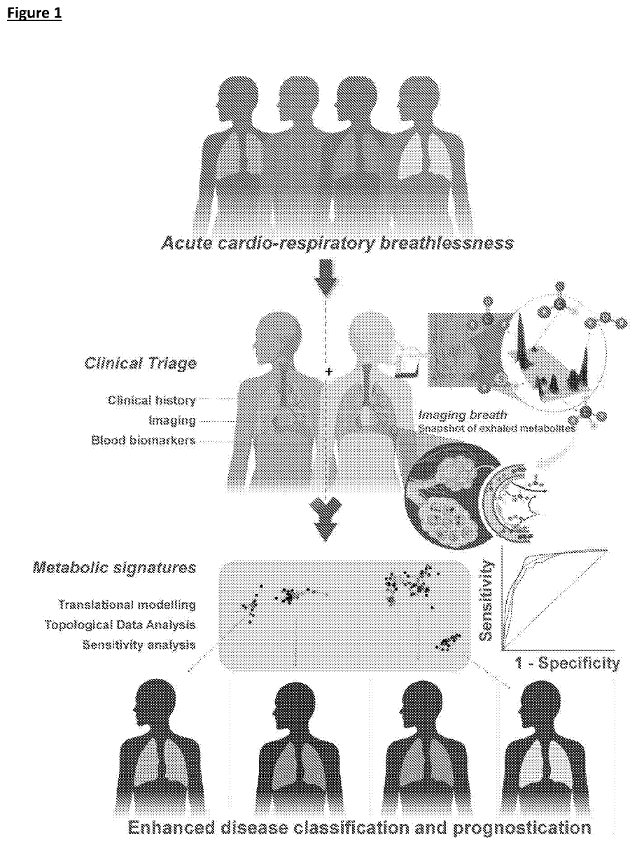

FIG. 1 is a visual abstract representing the proposed breath testing and diagnostic pipeline. Acutely breathless patients with cardio-respiratory disease exacerbations are currently triaged on admission by means of clinical assessment, digital pathology, and blood biomarkers. Lower airway derived breath volatile organic compound biomarkers, visualised using state of the art GCxGC mass spectrometry, undergo a process of chemometric and translational modelling coupled. The resultant breath metabolic signatures, provide accurate disease classification in acute cardiorespiratory patients, with co-location of specific VOC profiles and VOC classes with individual exacerbation subgroups.

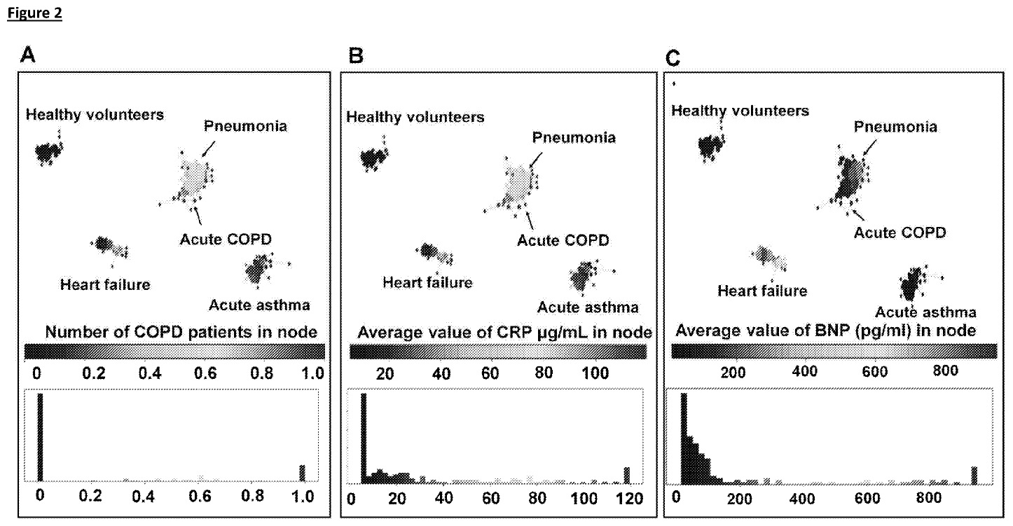

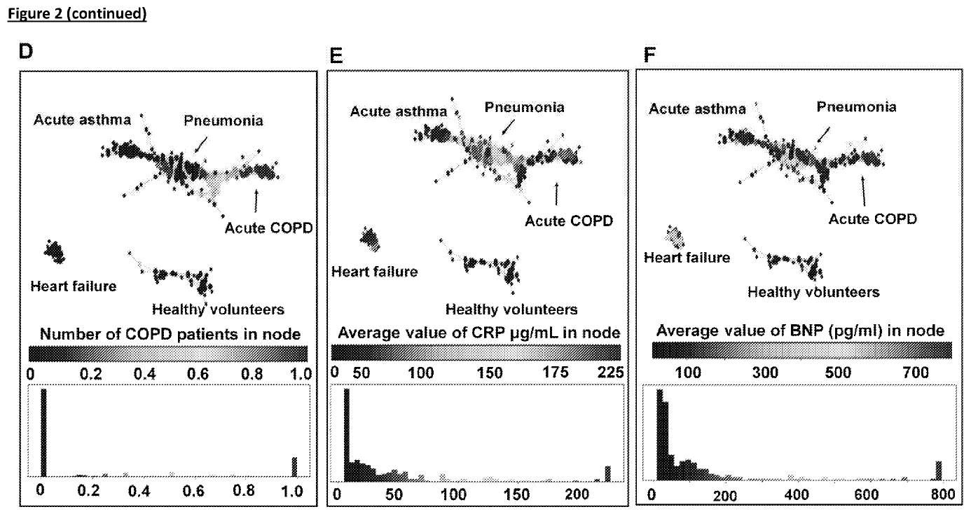

FIG. 2 is a topological Data Analysis (TDA) representing the various acute disease groups annotated by blood biomarkers. Each circle or ‘node’ in the TDA graph represents a subject or group of subjects. Similar subjects are grouped together in the same node and the relative similarity of the subjects is represented by the proximity of the nodes and the size of each node is determined by the number of subjects within it. (A) Visual mapping of the acute disease groups in the discovery cohort (n=139), based on the discriminatory 805 features and coloured by proportion of acute COPD exacerbations in each node. (B) The network is colour coded by the average values of CRP in each node in the discovery cohort (n=139). Higher CRP values corresponded topologically with the COPD and pneumonia patients. (C) The network is colour coded by the average values of BNP in each node in the discovery cohort (n=139). Higher BNP values corresponded topologically with the heart failure patients. (D) The network is coloured by proportion of acute COPD exacerbations in each node in the replication cohort (n=138). In replication cohort, Pneumonia and COPD exacerbation subjects occupied polar ends of the same TDA network. (E) The networks are coloured by the average values of CRP in each node. High CRP values corresponded topologically with the pneumonia subjects. (F) The networks are coloured by the average values of BNP in each node. High BNP values corresponded topologically with the heart failure subjects.

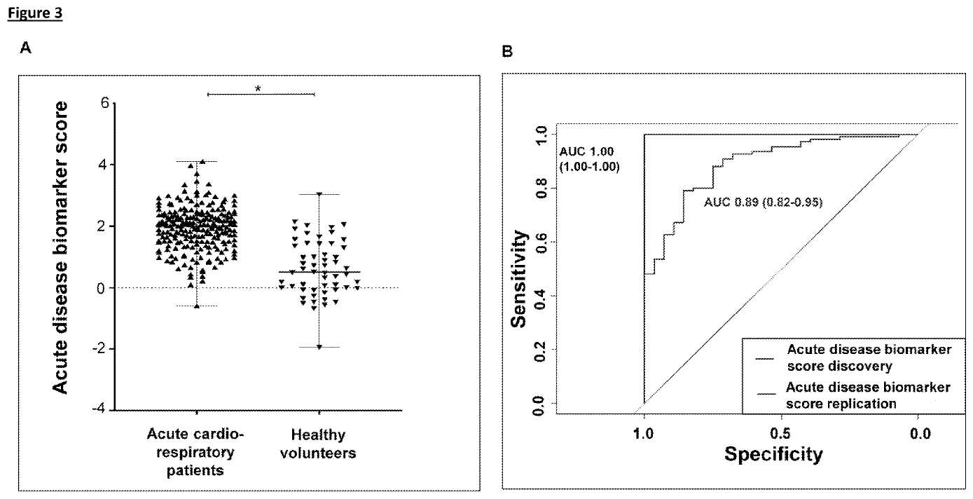

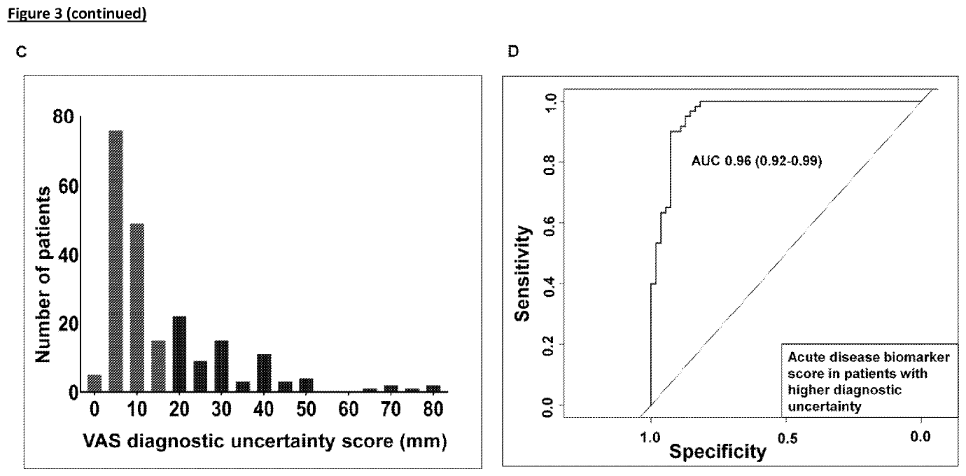

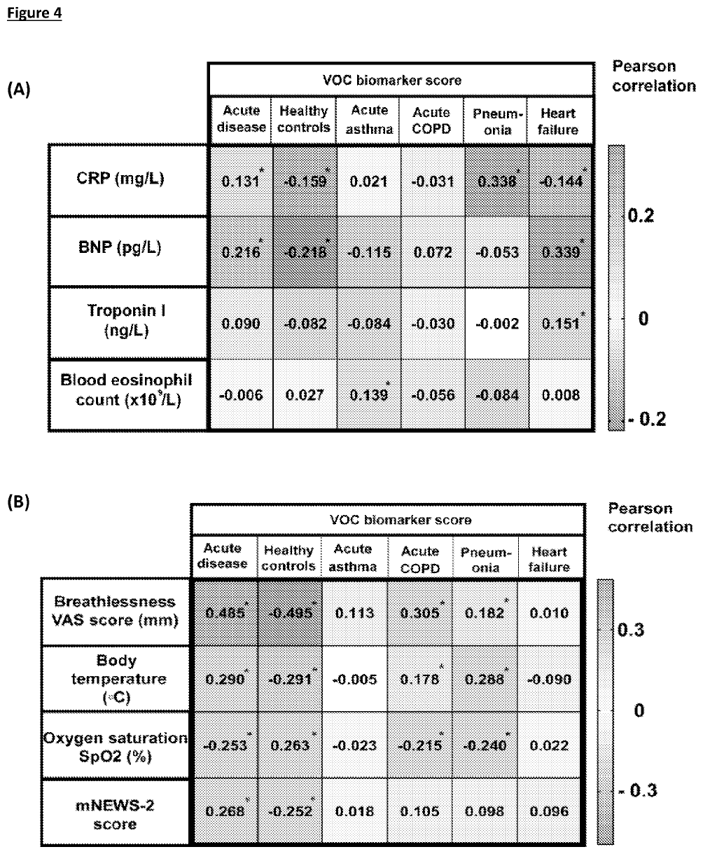

FIG. 3 is: (A) scatter plot demonstrating significant difference between breath VOC biomarker score values in acute cardio-respiratory patients compared to healthy volunteers. The black horizontal line within the scatter plot represents the median value of the biomarker score. Mann Whitney test p-value <0.0001. (B) Receiver operating characteristic (ROC) curve of participants in the discovery (black line)—AUC 1.00 (1.00-1.00), and replication cohorts (blue line)—AUC 0.89 (0.82-0.95) p<0.0001. (C) Histogram showing the number of patients with higher diagnostic uncertainty (blue bars with values >upper quartile value of 20 mm). (D) ROC curve assessing the discriminatory power of exhaled breath VOCs in participants with higher diagnostic uncertainty. AUC 0.96 (0.92-0.99) p<0.0001.

FIG. 4 is: (A) a Pearson's correlation of disease-specific VOC scores and blood-based biomarkers. Pearson correlation demonstrating the positive and negative correlations between breath VOC scores and blood-based biomarkers. * Significant correlations, p-value <0.05; and (B) a Pearson's correlation of disease-specific VOC scores and admission observations. Pearson correlation between the VOC biomarker score and admission vital signs. VAS: Visual Analogue Scale (100 mm), participants were asked to rate their breathlessness on a 100 mm VAS on admission.

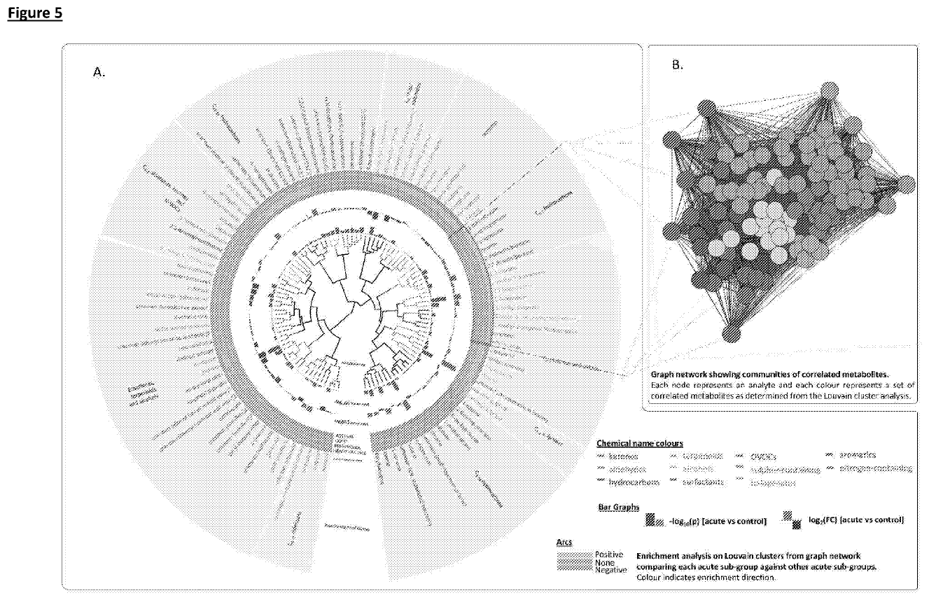

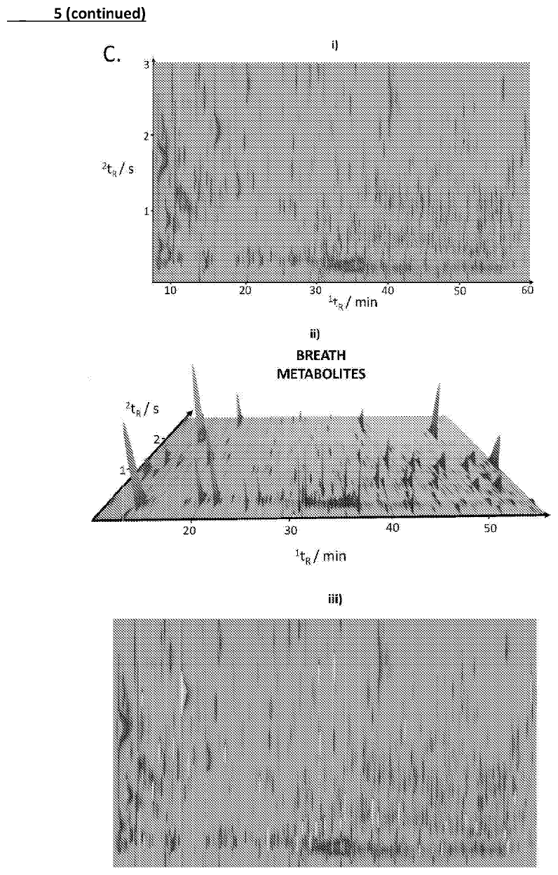

FIG. 5 is: (A) a circular correlation tree generated based on metabolite set enrichment and chemical similarity analysis on of 101 breath volatiles associated with acute breathlessness. Branches depict metabolite sets derived using the ChemRICH (Methods) bar graphs portray −log₁₀ (p) and log₂ (fold change) values of 101 features extracted using LASSO regression FIG. 16 in acute breathlessness compared with control group. The arcs represent the Louvain clusters, derived from the correlation graph (green for upregulated, red for not significant, blue for downregulated according to K-S test result). Chemical names are coloured based on their chemical classification and coloured regions used to summarise broader chemical groups; and (B) a correlation graph showing metabolite communities identified using Louvain clustering, with the identity and location of the cluster significantly enriched in heart failure, projected onto the circular dendrogram. (C) i) Example GCxGC chromatogram showing complex profile of breath metabolites, ii) 3D render of chromatogram showing visualisation of breath markers and iii) phenotypic differences based on features included in the risk scores FIG. 16 (yellow, asthma: red, pneumonia; magenta, COPD: cyan, heart failure).

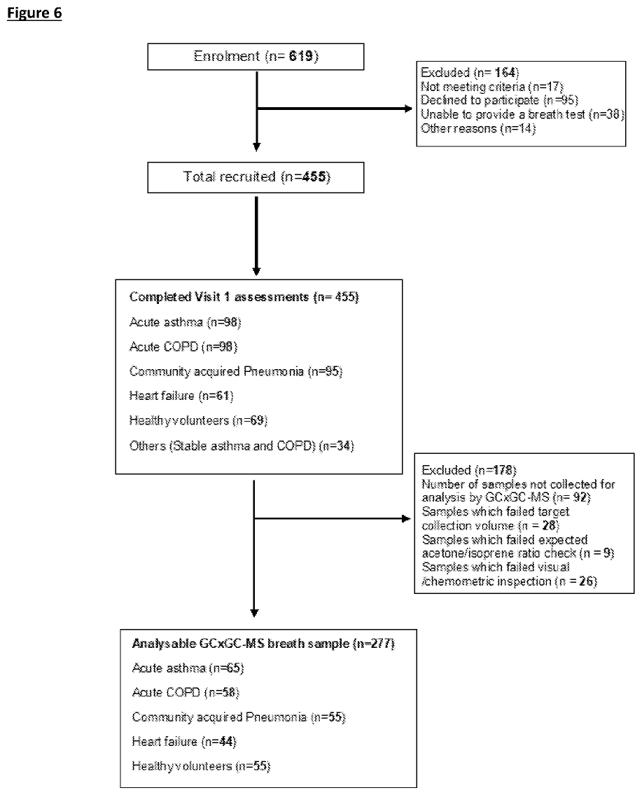

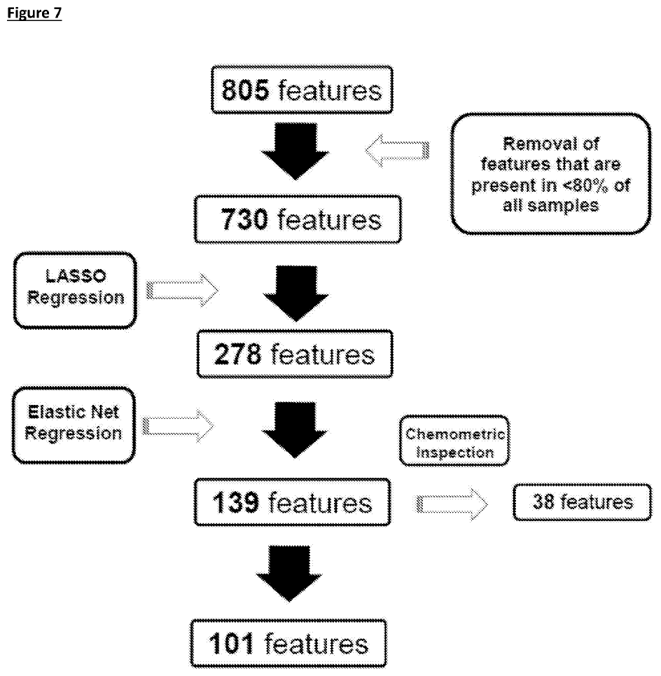

FIG. 6 is a consort diagram outlining the acute study recruitment and number of analysable GCxGC-MS breath samples.

FIG. 7 is a flow chart demonstrating the removal of exhaled breath features from 805 to 101. Least Absolute Shrinkage and Selection Operator (LASSO) and Elastic Net regularized regression models were adopted as the feature selection methods of choice owing to the high variables to subject ratio and the potential correlations among the candidate features

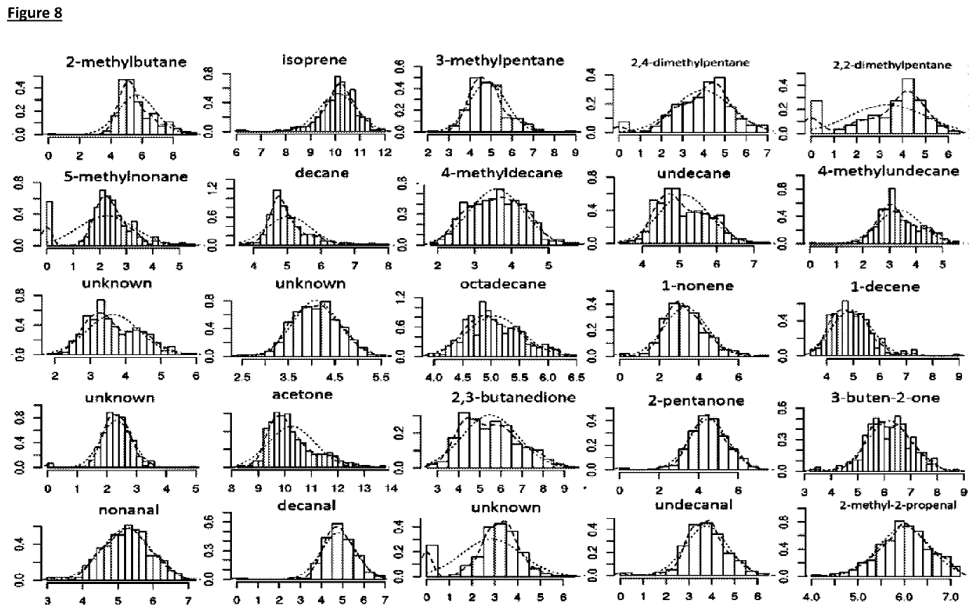

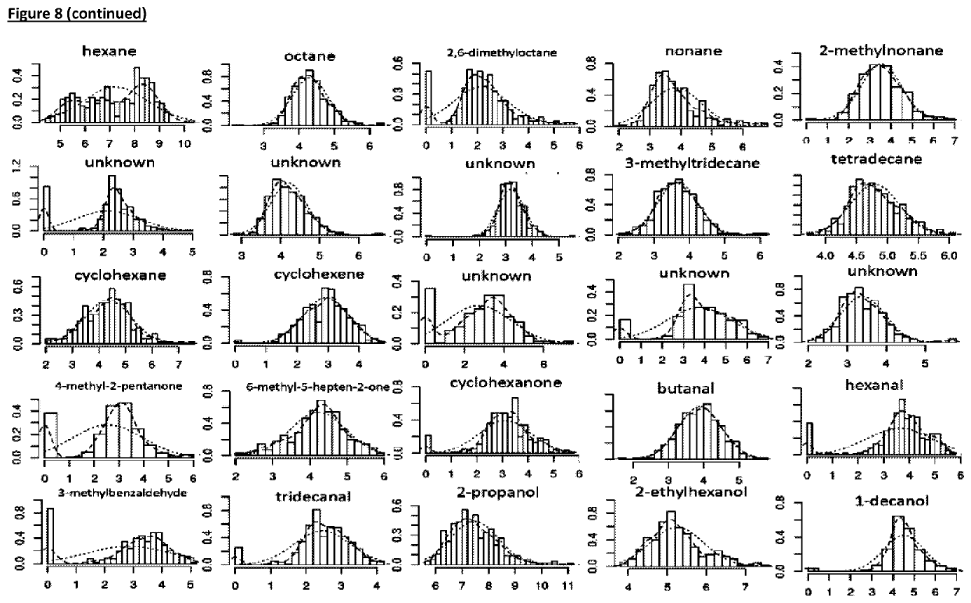

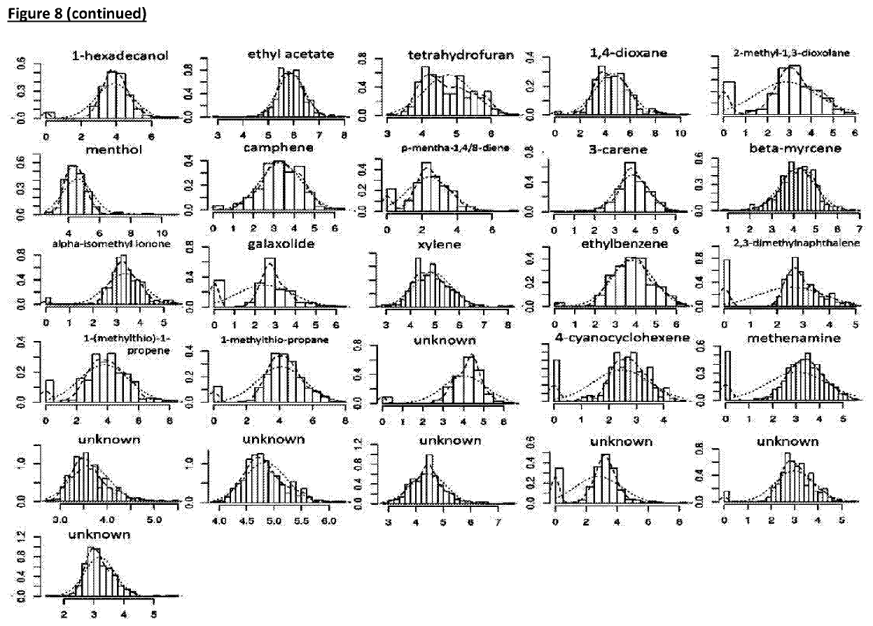

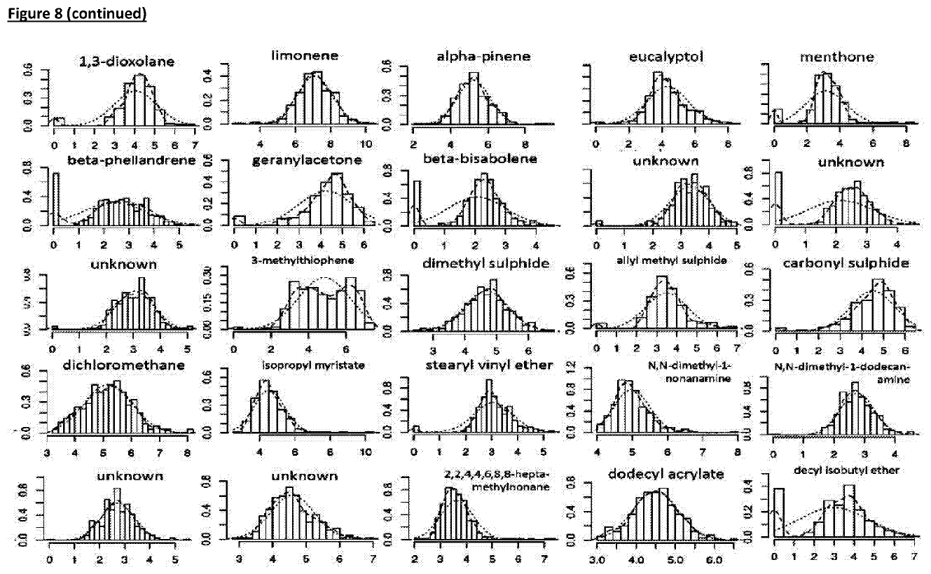

FIG. 8 is a graphical probability distribution of the final 101 exhaled breath features in the GCxGC-MS peak table. The features largely follow a similar distribution. Some features contained a mixture of zero and non-zero values, which have arisen owing to the measurement being below the instrument's lower limit of detection. Constant features (all zero values) were removed prior to fitting the main model.

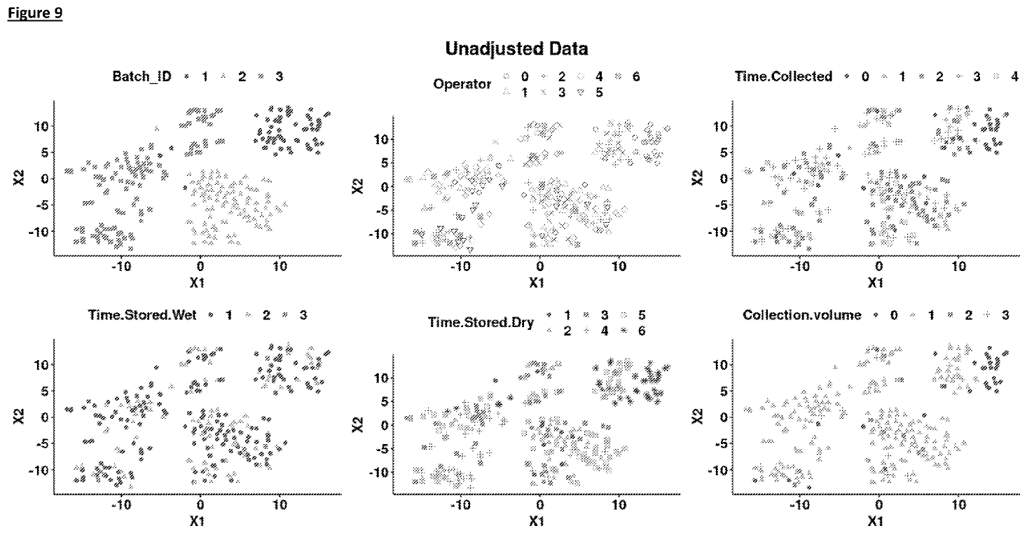

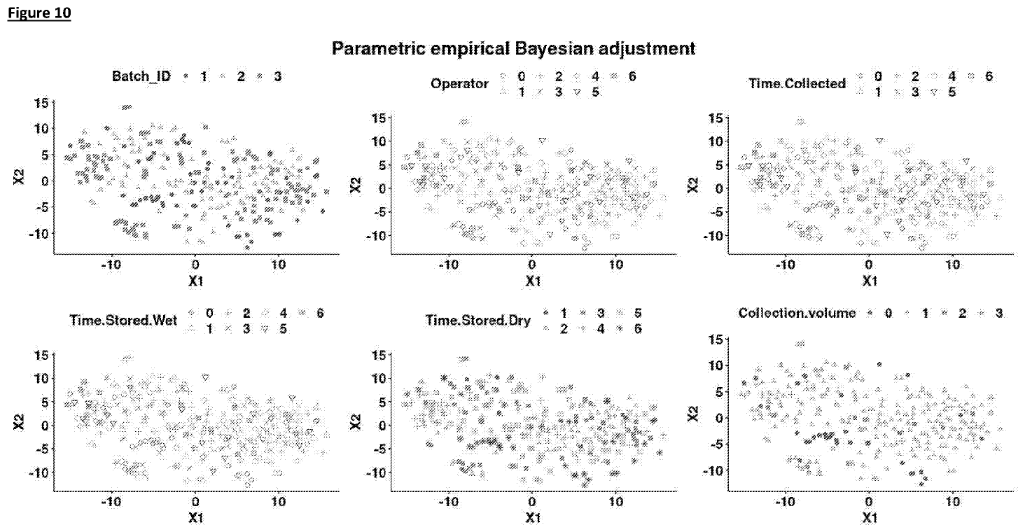

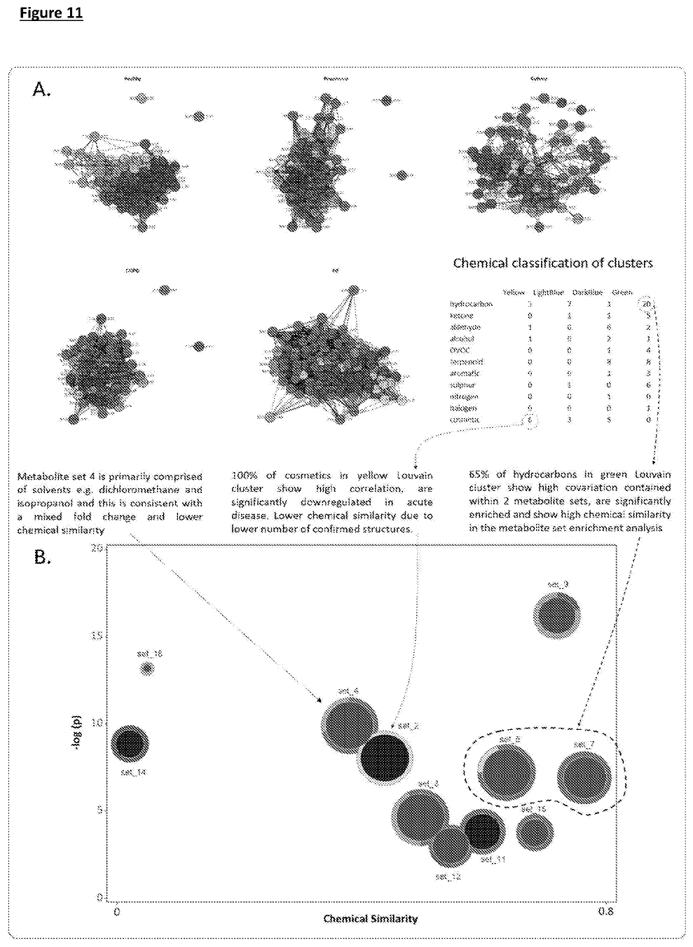

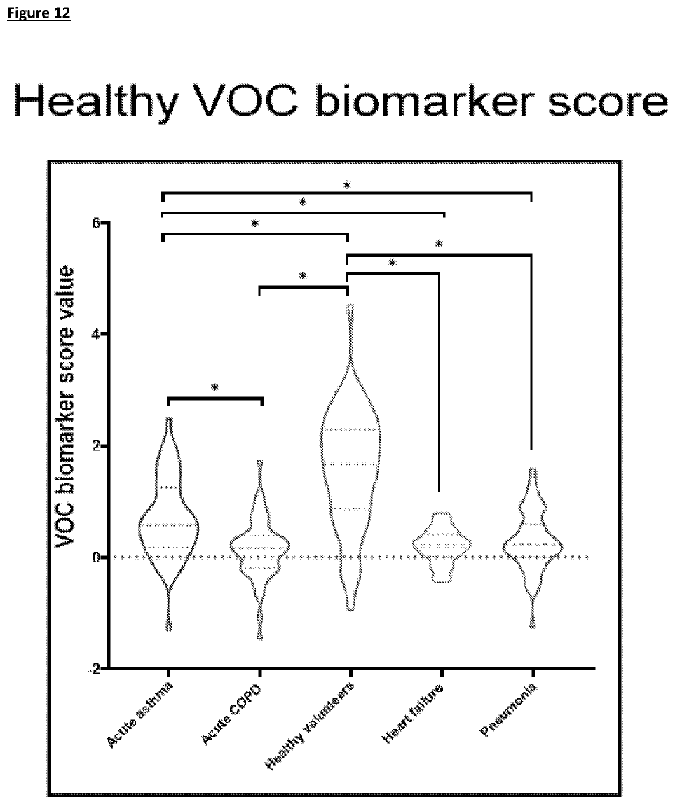

FIG. 9 is a 2-dimensional visualization of the high dimensional peak table before adjustment for batch effects. Clustering by date of collection ‘Batch_ID’ in the first panel can be clearly seen, compared to other variables (operators, time of collection, time of wet and dry storage, and collection volume) where no batch effects are apparent.

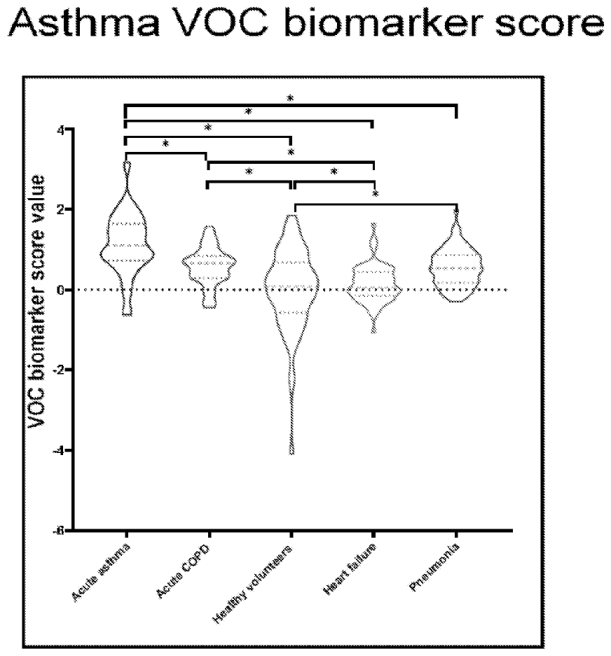

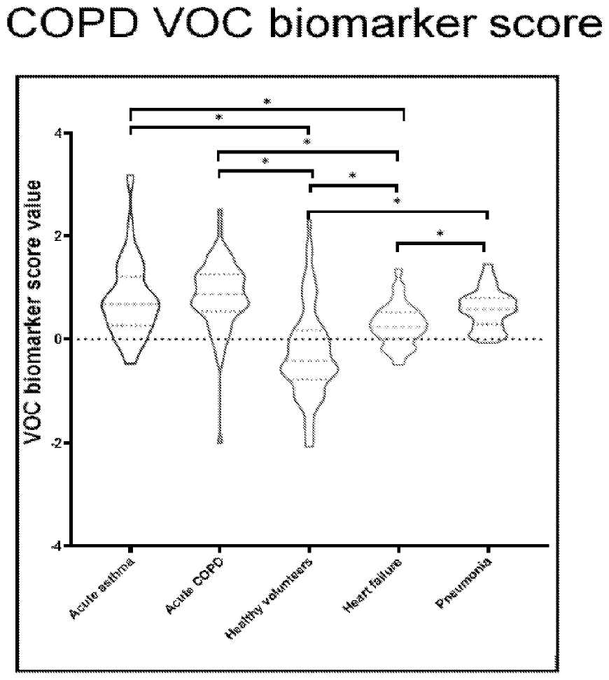

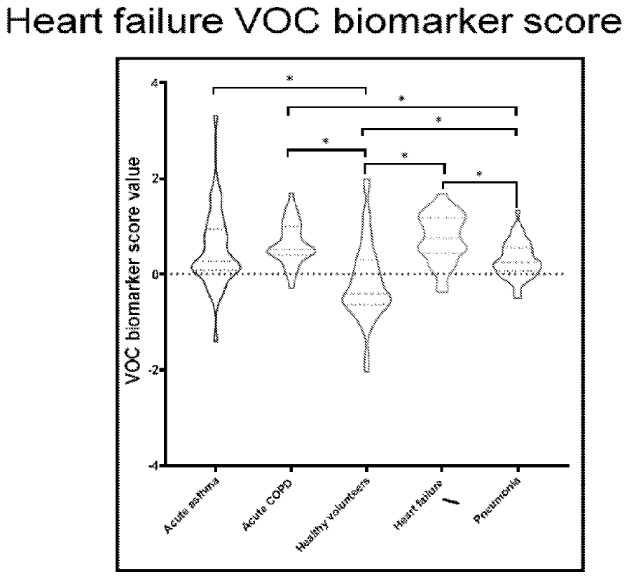

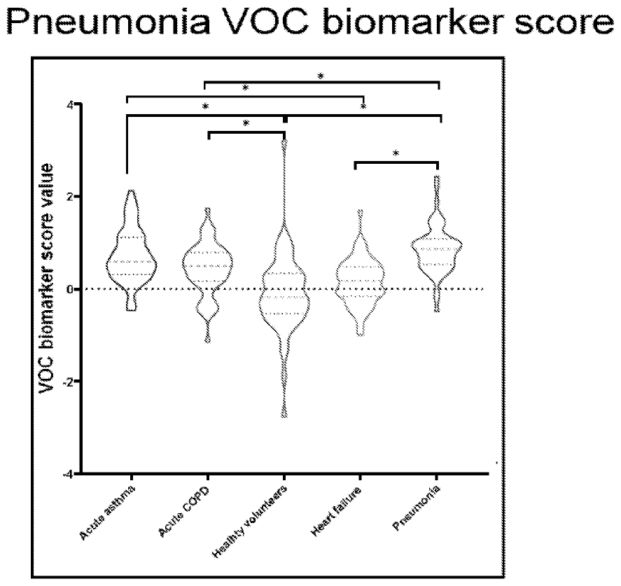

FIG. 10 is a 2-dimensional visualization of the high dimensional peak table after adjustment for date of collection ‘Batch_ID’. Clustering is no longer visible following parametric empirical Bayesian adjustment. FIG. 11 is A) Correlation graphs showing how the breath metabolites (panel of 101) are correlated within each of the casual subgroups, coloured based on Louvain clusters to highlight differences across the networks. Visual differences highlighted include the green Louvain cluster, being highly compact in the control group and dispersed in the acute groups. B) Output of the ChemRICH analysis, showing metabolite sets (circles) significantly enriched during acute breathlessness (size indicative of fold change; red=upregulated; blue=downregulated). The upregulated metabolite sets with high chemical similarity (based on Tanimoto coefficient) consisted predominantly of acyclic and branched hydrocarbons, belonging to the green Louvain cluster (indicated by outer ring colour). The quantitative output of the ChemRICH analysis complements the visual differences in the graph networks

FIG. 12 shows violin plots demonstrating significant differences between VOC biomarker scores values across the different disease sub-groups. * Kruskal-Wallis test comparing non-parametric data. * Significant p value<0.0001.

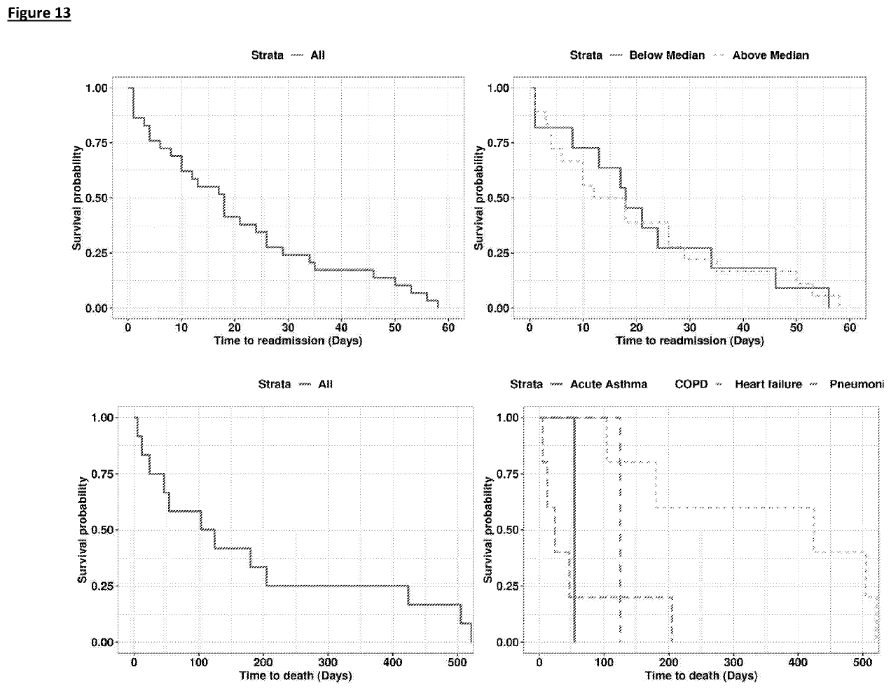

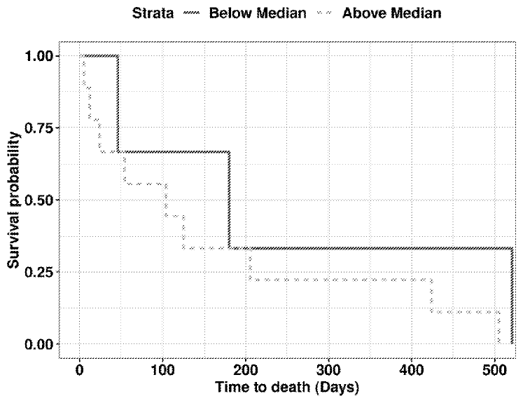

FIG. 13 shows a Kaplan-Meier survival analysis. (A) Total of 29 patients were readmitted within 60 days of hospital discharge. (B) Total number of patients readmitted classified by their acute disease VOC score median value, showing no significant difference in the readmission rate based on the underlying VOC score p value of 0.77 (log−rank test for equality f survivor function). (C) Total of (n=12) deaths in the 2 years follow-up period (D) Kaplan-Meier survival analysis for all-cause 2 year mortality, classified by disease groups. (E) Kaplan-Meier survival analysis for all-cause 2 year mortality, classified by acute disease VOC score median value.

There was no significant difference between groups, p value of 0.07 (log−rank test for equality f survivor function)

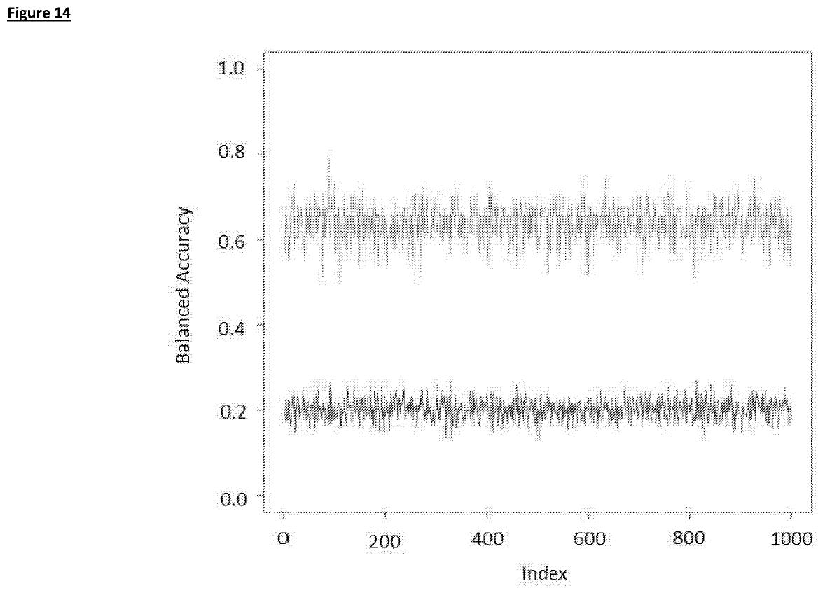

FIG. 14 is a graph that demonstrates the overall classification accuracy using all 5 biomarker scores.

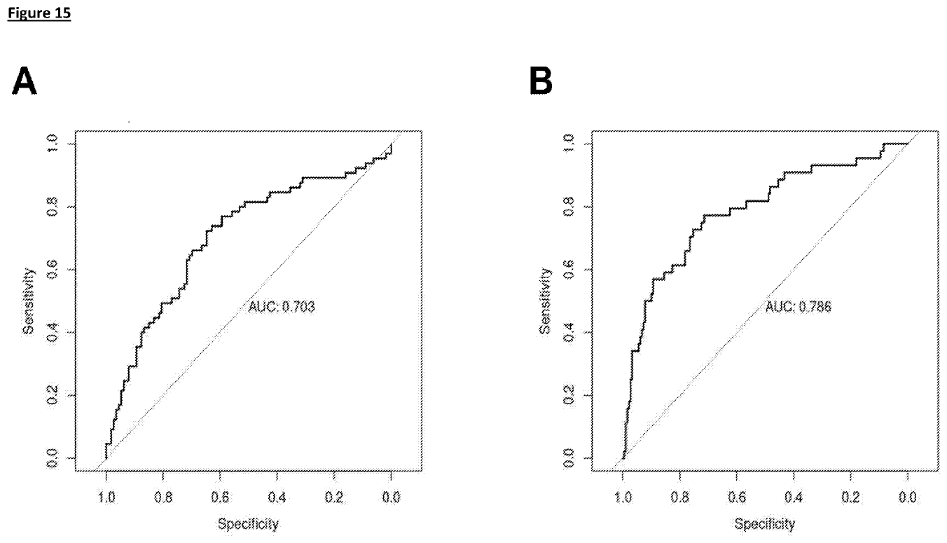

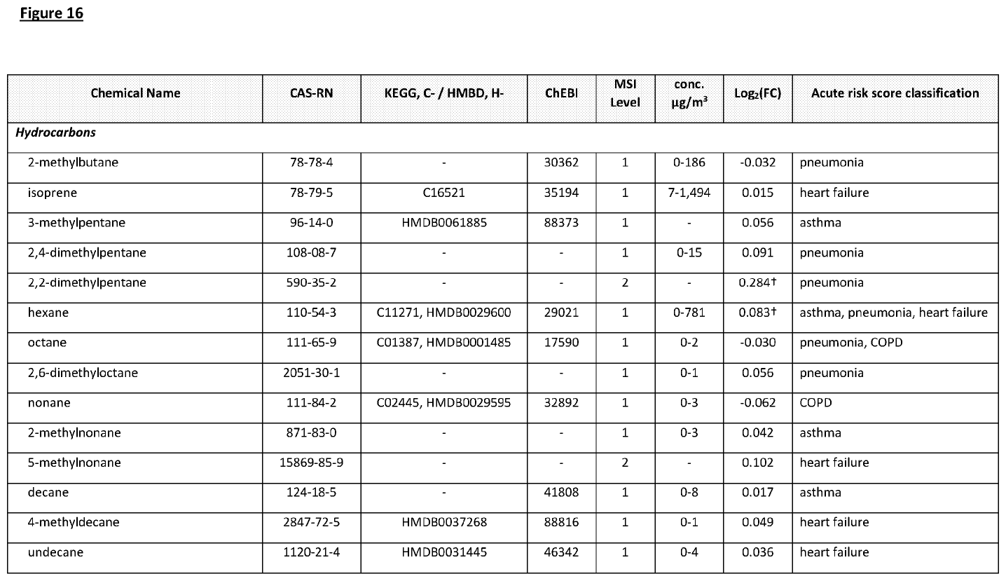

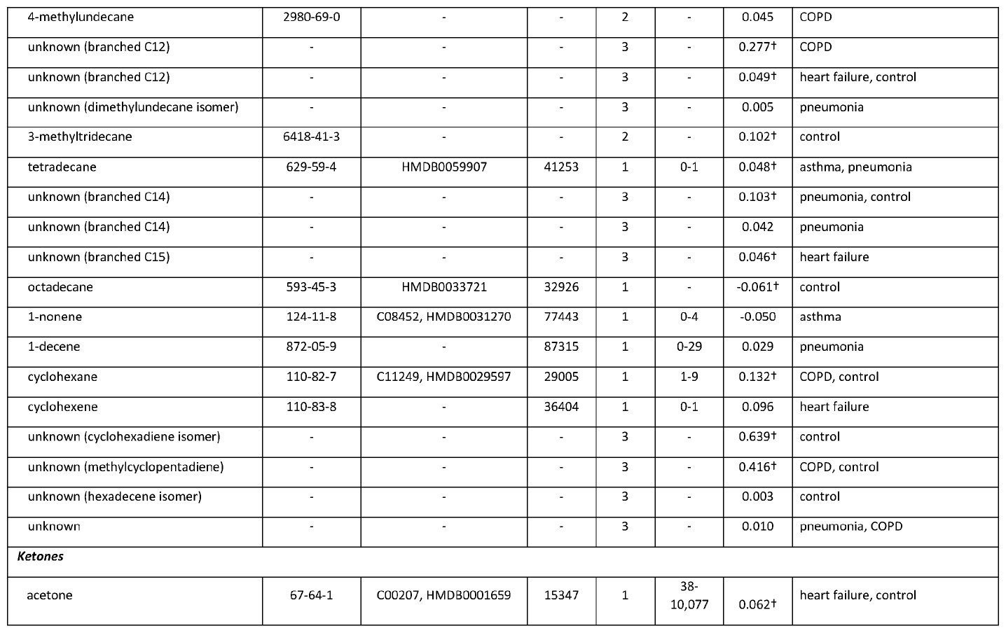

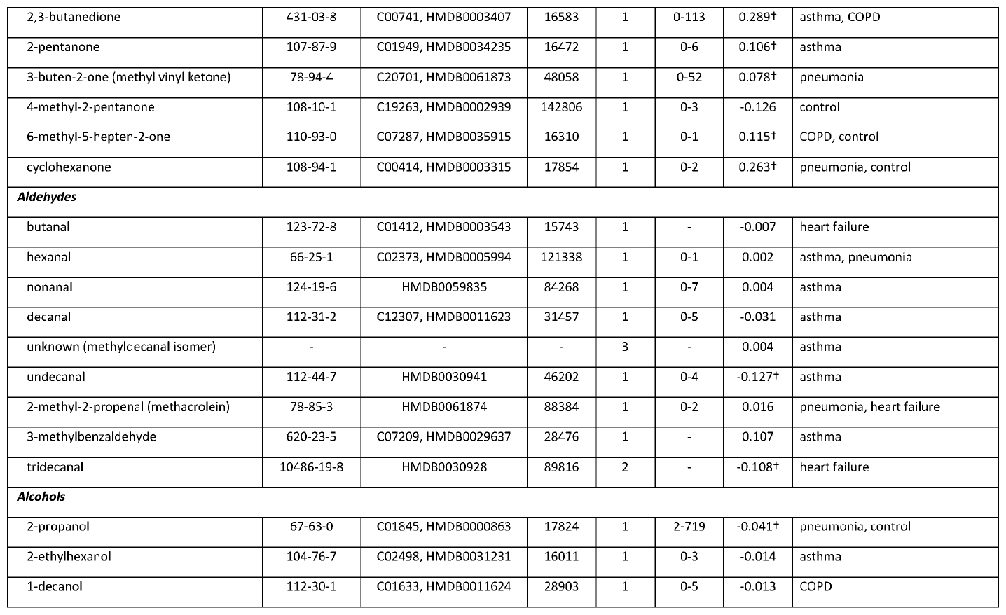

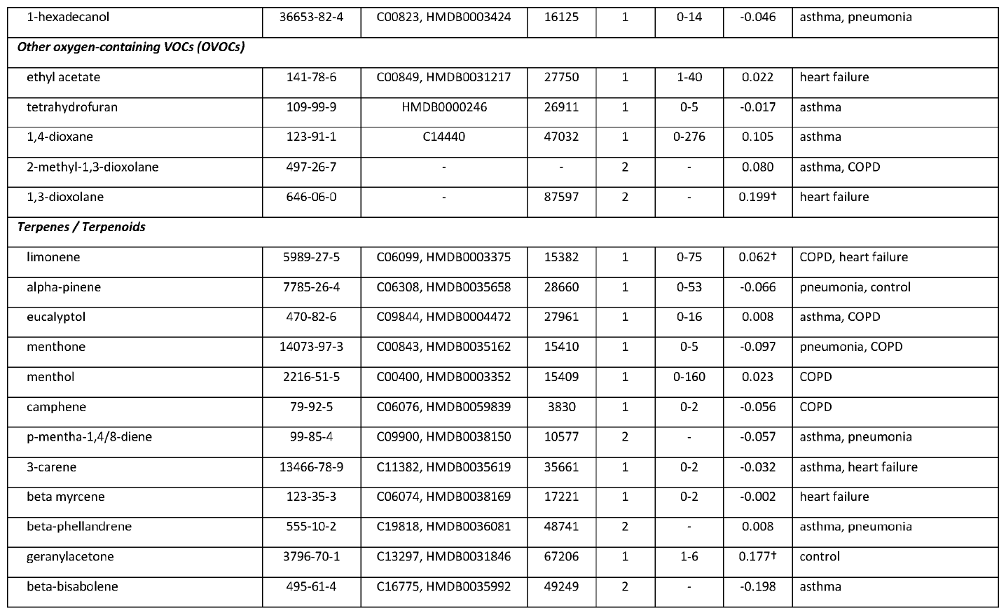

FIG. 15 is (A) a comparative ROC analysis demonstrating the diagnostic value of asthma VOC score against the predominantly infection-driven acute disease groups (pneumonia and COPD) in the pooled (discovery and replication) cohorts. (B)

Comparative ROC analysis demonstrating the diagnostic value of heart failure VOC score against other acute disease subgroups (asthma, COPD and pneumonia) in the pooled cohorts.

FIG. 16 shows the chemical assignment of selected predictive markers from the regression model detailing chemical name, CAS registry number, KEGG, Human Metabolome Database and ChEBI identifiers and MSI-compliant metabolite identification level, concentration range and fold change (expressed as log2) between acute and control groups, and compound contribution towards disease-specific biomarker risk scores (†adjusted p-value<0.05).

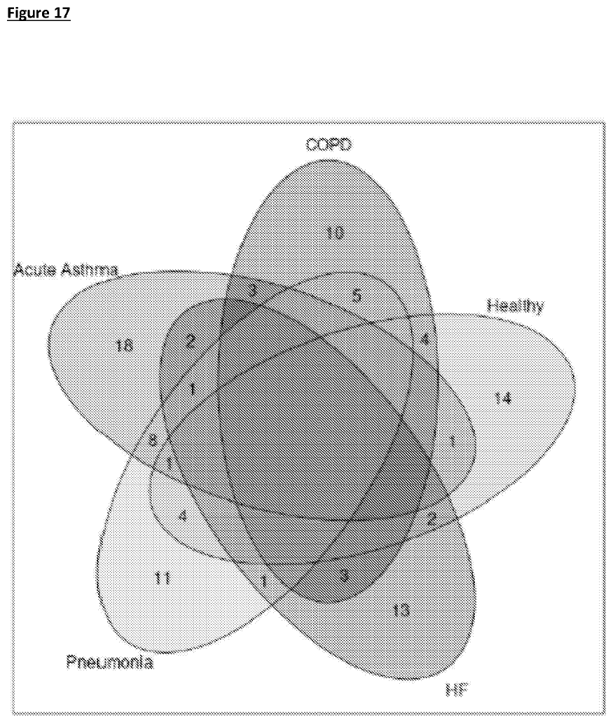

FIG. 17 is a Venn diagram demonstrating the distribution of the final panel of 101 exhaled 362 breath biomarkers across the different disease groups.

### EXAMPLES

Disclosed herein is a real-world, prospective study of acutely unwell hospitalised patients presenting with breathlessness due to severe exacerbations of cardio-respiratory aetiology (asthma, chronic obstructive pulmonary disease (COPD), heart failure or pneumonia) and healthy controls. It has now been demonstrated that breath biomarkers can reliably and repeatedly identify acute cardio-respiratory breathlessness; including in the presence of diagnostic uncertainty.

### Methods

Trial design, participants and ethical approval

The clinical study was a prospective, real-world, observational study, carried out in a tertiary cardio-respiratory centre in Leicester, United Kingdom. Participants were recruited all year-round from May 2017 through to December 2018.

Patients with self-reported acute breathlessness, requiring admission and/or a change in baseline treatment, presenting within University Hospitals of Leicester (UHL) were approached for study participation. Following triage and senior clinical assessment, if a primary clinical diagnosis of (i) acute decompensation of heart failure, (ii) exacerbation of asthma/COPD, or (iii) adult community acquired pneumonia was suspected by the triage nurse/attending clinician at triage, members of the research team would evaluate patients against predefined eligibility criteria for study participation. Informed consent was obtained in all participants within 24 hours of hospital admission. Age and/or home environment matched healthy volunteers were recruited. Where environment-matched controls were unsuitable, healthy volunteers were recruited from local recruitment databases and via advertising. Further details of healthy volunteers' comorbidities and medication use are outlined in Table 1.

The trial was conducted in accordance with the ethics and principles of the deceleration of Helsinki and Good Clinical Practice Guidelines. All patients provided written consent. The National Research Ethics Service Committee East Midlands has approved the study protocol (REC number: 16/LO/1747). Integrated Research Approval System (IRAS) 198921.

TABLE 1
demonstrates comorbidities and medications used by
study participants, classified by disease and health.
Values expressed as N (%). Table includes
comorbidities occurring in >5% of participants
and medications used by >5% of participants.

| | Healthy | Acute disease |
|---|---|---|
| Comorbidities | controls N (%) | group N (%) |
| Anxiety/Depression | 5 (9) | 26 (11.7) |
| Diabetes Mellitus | 4 (7.2) | 45 (20) |
| Essential hypertension | 16 (29) | 58 (26) |
| Ischaemic heart disease | 4 (7.2) | 13 (5.8) |
| Arthritis | 5 (9) | 12 (5.4) |
| Thyroid disorder | 3 (5) | 15 (6.7) |
| COPD | 0(0) | 58(26) |
| Asthma | 0(0) | 65(29) |
| Heart failure | 0(0) | 44(19.8) |

Medications

| | | |
|---|---|---|
| Inhaled therapies | | |
| Salbutamol | 1 (1.8) | 153 (68) |
| ICS/LABA | 0 (0) | 53 (23) |
| Lipid lowering agents | | |
| Atrovastatin | 9 (16) | 57 (25.6) |
| Simvastatin | 7 (12) | 23 (10) |
| GORD medications | | |
| Lansoprazole | 9 (16) | 49 (22) |
| Blood pressure lowering | | |
| agents | | |
| Amlodipine | 5 (9) | 25 (11.2) |
| Lisinopril | 4 (7) | 11 (5) |
| Ramipril | 8 (14) | 37 (16.6) |
| Antidepressants | | |
| Citalopram | 3 (5) | 13 (6) |
| Sertraline | 3 (5) | 15 (6.7) |
| Thyroid medications | | |
| Levothyroxine | 5 (9) | 15 (6.7) |
| Cardiac medications | | |
| Aspirin | 5 (9) | 36 (16.2) |
| Analgesics | | |
| Paracetamol | 6 (10) | 103 (46) |

Recruitment started in February 2017 and following analytical method development and optimisation of a robust sample pathway for achieving continual deployment, collection and analysis of sorbent tubes to-and-from clinic, the analysis of samples by GCxGC-MS was set up and brought online later that year (August 2017). The denominator for the entire study was 455 participants and for the GCxGC-MS study presented here is 363 participants, with a 76% GCxGC-MS completion rate (FIG. 6).

A detailed Survival analysis of study participants is demonstrated in FIG. 13.

The trial was conducted in accordance with the ethics and principles of the deceleration of Helsinki and Good Clinical Practice Guidelines. All patients provided written consent. The National Research Ethics Service Committee East Midlands has approved the study protocol (REC number: 16/LO/1747). Integrated Research Approval System (IRAS) 198921.

Clinical adjudication

A clinical adjudication process was introduced to precisely define and quantify the diagnostic labels in the study, addressing any potential misclassification. A panel of two senior clinical adjudicators (SS & NG) reviewed all available case notes, imaging and determined the primary diagnosis for each case by discussion to reach a concordance. The degree of diagnostic uncertainty was marked on a 100 mm visual analogue scale (VAS scale), blinded to given diagnosis and blood biomarkers.

The process was implemented with emphasis on mirroring an acute triage pathway, where all pathology data required to support the diagnosis e.g. CRP, BNP are not available at the initial clinical review.

The degree of diagnostic uncertainty obtained from the clinical adjudication process was factored into the block randomisation and subjects with higher diagnostic uncertainty (≥upper quartile=20 mm) were assessed separately as previously described (FIG. 3c-d).

Collection of breath samples

Exhaled breath collection was attempted in all consented participants using a CE marked breath sampling device ‘Respiration Collector for In Vitro Analysis’ RECIVA® (Owlstone Nanotech Ltd), in combination with a dedicated clean air supply unit. The ReCIVA® device aims to standardise the collection of alveolar breath by providing the patient with a VOC-clean air supply: controlling the flow, volume and fraction of breath collected, while directly sampling the exhaled VOCs onto the sorbent tubes. The ReCIVAR settings mode was set to ‘lower airways only’, the continuous monitoring of the CO₂ and partial pressure allowed targeting the VOC-enriched alveolar fraction of breath. The collection volume, flow rate and maximum sampling time were set to 1 L, 250 mL min⁻¹, and 900 seconds respectively. Breath sampling was well tolerated by all participants.

At the time of sampling, the room air and air supply were also sampled as environmental controls. This involved attaching a sorbent tube to a handheld personal pump (Escort Elf, Sigma Aldrich, Dorset, UK) and having the sampling end either open to the room air or attached to the ReCIVA® air supply line via a T-piece. 1 L of air was collected in total at a flow rate of 0.5 L min⁻¹ for 2 min.

Sorbent tubes were immediately capped (brass caps, Markes International Ltd) and placed in a fridge at 4° C. before being dispatched to the laboratory within 72 h. In an attempt to minimise background variation, sample collection was completed, when possible, in the same treatment room attached to the admissions ward. Unwell patients and those requiring supplemental oxygen, however, had their samples collected by their bedside.

Sample storage and preparation

Samples were dry purged on arrival for 2 min using nitrogen (CP grade with inline trap, BOC, Leicester, UK) at a flow rate of 50 mL min⁻¹ and then stored in the fridge at 2° C. until analysed. Before analysis, samples were left to reach room temperature before being spiked with a 0.6 μL aliquot of 20 μg mL⁻¹ standard solution containing deuterated toluene and octane, into a flow of nitrogen at a flow rate of 100 mL min⁻¹ for 2 min, purging the excess solvent.

Analysis of Room Air and Air Supply samples

Two separate elastic net regression models were fitted to peak tables for room air and air supply samples, both peak tables where logₑ(x+1) transformed and adjusted for batch effects (collection date) using PEBA. The independent variables were the final set of 101 features and the dependent variable was clinical diagnosis (Acute Asthma, Acute COPD, Pneumonia, Heart Failure or Healthy volunteers). After repeating 10-fold cross validation 100 times for each of the two models, only two features were found to have stable non-zero regression coefficients. These features were for air supply, a component of the pneumonia score and for room air, a component of the healthy score, highlighting the robustness of the selected feature separation models.

Exhaled Breath analysis

### TD-GCxGC-FID/MS

Breath samples were analysed by thermal desorption with comprehensive two-dimensional gas chromatography (GCxGC) using flow modulation and coupled to dual flame ionisation detection and mass spectrometry (MS). Dual detection, with the use of MS and flame ionisation detection (FID), utilises the excess flow from the flow-based modulator suited for volatile analyses, providing both quantitative and qualitative results.

Analysis by GCxGC was optimised and conducted using an Agilent 7890A gas chromatogram, fitted with a CFT flow modulator and 5799B mass spectrometer with a high efficiency EI ion source (Agilent Technologies Ltd, Stockport, UK). The instrument was coupled to a TD-100xr thermal desorption auto-sampler (Markes International Ltd, Llantrisant, UK). Samples were analysed in trays: typically six per tray along with a reference mixture containing n-alkanes and aromatics run every tray and a reference indoor air VOC mixture run every four trays. Data was acquired in MassHunter GC-MS Acquisition B.07.04.2260 (Agilent) and processed (i.e. baseline correction, alignment, feature extraction) with a workflow previously developed and optimised, using GC Image™ v2.8 suite (GC Image, LLC. Lincoln, NE, US) and Python. The sorbent tubes used were Tenax/TA with Carbograph ITD (Hydrophobic, Markes International Ltd) with matching cold trap. Chromatographic features arising from analytical artifacts were removed from the peak table (e.g. ubiquitous siloxanes).

For purposes of quality control, samples were analysed using a detailed sample history, metadata and experimental data were recorded at every stage of the collection and analysis using the open-access LabPipe toolkit.

Chemical speciation of identified breath biomarkers

The chemical nature of volatile metabolites exhaled in breath comprises a diverse mixture of non-novel, low-molecular weight compounds. Thus, for the majority of features, chemical identification involved comparison with an authentic reference compound in accordance with the Metabolomics Standard Initiative (MSI) Level 1 criteria for metabolite identification (FIG. 16). Identification was based on a minimum of two independent and orthogonal identifiers including primary and secondary retention time, mass spectral similarity match and calculated retention index. When an authentic reference compound was unavailable, chemical identification was compliant with MSI Level 2 for putative annotations. The highly structured chromatographic data and group-type separation afforded by GCxGC, alongside a well-characterised chromatographic space from analysing an extensive library of authentic compounds, gave increased confidence in the tentative assignments made. The orthogonal separation of GCxGC also meant chemical identification of unknown metabolites could be made, at minimum, in compliance with MSI Level 3 for putative chemical classification.

The diagnostic accuracy of the reported exhaled breath VOCs was tested following the Standards for reporting of Diagnostic Accuracy Studies guidelines; and for multivariate prediction models, Transparent Reporting of multivariate prediction model for Individual Prognosis or Diagnosis (TRIPOD) was followed.

Quality control and quality assurance systems

A number of traceable and verifiable quality control and quality assurance (QC/QA) procedures have been applied throughout the breath sampling and analysis steps. This ensured efficient prevention of any anticipated defects and high deliverable standards. In order to eliminate any samples from the final analysis that were of poor quality four criteria were used to selected for high quality breath samples. These were:
1. 24 800 mL of breath collected from the patient to ensure sufficient pre-concentration of trace VOCs present in breath.
2. The concentration of isoprene and acetone in the air supply were ≤3 standard deviations of the mean air supply concentration. This ensured that no breath samples were mis-assigned as air supply samples.
3. The concentration of isoprene and acetone in breath were ≥10 and ≤5 standard deviations, respectively, above the levels measured in the patient air supply. This ensured that the samples were not mis-assigned air supply samples, and that breath had been collected onto the sorbent tubes
4. The chromatogram, on visual review, was not distorted by an abundance of exogenous compounds (i.e. overloaded peaks).

The number of breath samples fulfilling all QC/QA criteria is outlined in (FIG. 6).

Sample analysis QC/QA procedures

For purposes of quality control, samples were analysed in accordance with a previously published workflow and a detailed sample history, metadata and experimental data were recorded at every stage of the collection and analysis using the open-access LabPipe toolkit. The chromatographic method was optimised for peak shape, sensitivity and separation; quality control charts of the internal standards were used to track the stability of the TD-GCxGC-FID/MS analysis, and instrument performance was evaluated following the assessment of the variation of retention times, peak area and shape of VOCs in two standard reference mixtures every six samples. Before being conditioned and sent to clinic, the number of heat cycles and weight for each tube was recorded to monitor tube age and integrity. For each conditioning cycle, all tubes were given a batch number and a batch blank was analysed to monitor contamination from the beginning of the sample preparation process. Furthermore, all batches were given an expiry of two weeks to ensure routine monitoring. To minimise the influence of biological and analytical confounders (e.g. circadian rhythm, sample stability), potential effects due to the operator, date of analysis, time of day collected, storage time before dry purging, sample storage time after dry purging and collection volume were assessed and where necessary accounted for in the batch correction. In addition to the routine analysis of reference standards, used to monitor retention shift and instrument response, the TD-GCxGC analytical system underwent a programmed heat cycle between each sample to reduce potential issues arising from sample carry-over, and a TD-trap blank and empty sorbent tube were analysed every six samples to monitor the instrument baseline signal.

Statistical procedures

Statistical analysis was performed using R (3.6.1 and 4.0.0, R Core Team (2019). This research used the SPECTRE High Performance Computing Facility at the University of Leicester. Baseline data and figures were presented as mean ± (SD), and median (IQ range). Data was analysed using (ANOVA) to assess the differences between groups for normally or approximately normally-distributed variables and Kruskal-Wallis for non-normally distributed variables. Pearson chi-squared and Fisher's exact were used to assess the differences in categorical variables. All P values are two sided and significant at the 0.05 level, unless reported otherwise. Study sample size calculations were informed based on sample size estimation for adequate sensitivity and or specificity (Sample size estimation section).

Discovery and replication sets

The 277 subjects were randomised post-hoc to Discovery and Replication cohorts in a 1:1 ratio through block random assignment. Randomisation was stratified based on (I) adjudicated clinical diagnosis, (II) time to breath-testing from the point of hospital admission, and (III) clinical diagnostic uncertainty score. The R package randomizer was used to perform block random assignment. After block randomisation there were 139 and 138 subjects in the discovery and replication sets respectively.

Examination of topological equivalence in the discovery and replication sets

Topological data analysis is an unsupervised machine-learning tool used for the analysis of large-scale, high-dimensional, complex datasets. It is highly sensitive to patterns that are often overlooked by other data reduction tools like Principal Component Analysis (PCA).

TDA captures the shape of data and provides a meaningful geometric representation where complex relationships within the data points are preserved and jointly considered.

Prior to performing TDA each feature was logₑ(x+1) transformed. TDA parameters were set as: number of hypercubes=20, where the number of hypercubes refers to the number of overlapping intervals of the projection. The distance between data points was measured using the Euclidean distance. The first two linear discriminant functions (LD1) and (LD2) were used as the projection. Clustering on the overlapping intervals on the projection was done using agglomerative (bottom up) hierarchical clustering with complete linkage. TDA was performed using Kepler Mapper 1.4.0 with Python 3.5.

Herein, the equivalence between topological data shapes generated using 805 volatile features extracted from the GCxGC-MS peak table was computed, in both the discovery and replication cohorts (FIG. 2).

Exhaled breath feature selection

Feature selection was implemented via Lasso and Elastic-Net Regularized Generalized Linear Models (GLMNET) using the glmnet package in R. After removing features present in <80% of all samples from the logₑ(x+1) transformed discovery GCxGC-MS peak table, 735 feature matrix was obtained. A multinomial regression model using LASSO regularization was fitted to the 735 feature matrix in the discovery set using 10 fold cross validation, with the dependent variable in the model being clinical diagnosis (Acute Asthma, Acute COPD, Pneumonia, Heart Failure or Healthy volunteers). The 10-fold cross validation was repeated 100 times, features that had a non-zero regression coefficient in more than 80 of the cross validation runs were considered as being stable candidate features predictive of the outcome (clinical diagnosis), and this resulted in 278 stable candidate features.

A multinomial regression model using elastic net regularization was fitted to the 278 features with the dependent variable in the model being clinical diagnosis. Following the chemometric inspection detailed above and the lasso and elastic regression analysis, a final set of 101 exhaled breath volatile compounds was generated (FIG. 7).

A multinomial regression model using elastic net regularization was fitted to the matrix of 101 breath biomarkers with the 10-fold cross validation repeated 100 times. The R package glmnetUtils was used to determine the optimal value of a the elastic net penalty, the best value for a was 0 (Ridge regression). Linear combinations of the most stable features from the multinomial regression model fitted to the 101 biomarkers formed a set of scores for predicting probability of belonging to the different disease groups (acute Asthma, acute COPD, pneumonia, heart failure or healthy volunteers). Ridge regression with a logit link function (binary logistic regression) was fitted to the 101 breath relevant features, the dependent variable was ‘acute disease’, as a binary outcome. The linear predictor from the combination of the most stable features was used to as a score to predict acute disease.

Co expression and feature enrichment analysis

It was of interest to investigate if within the final set of 101 features, sets of ‘co expressed’ features existed, i.e. sets containing features that are correlated. Considering sets of co-expressed features has value in terms of reducing the dimensions of a problem and mitigating the multiple testing problem through the use of enrichment score. Co expression and feature enrichment analysis are described in the (Supplementary Information).

Metabolite sets were derived based on Ward hierarchical cluster analysis using the ChemRICH method (FIG. 5A), and more broader communities were derived from Louvain cluster analysis to help interpret the correlation graphs (FIG. 5B, see Supplementary Information section on co-expression and feature enrichment analysis). Covariation among metabolites lacks evidential value on its own, therefore, set-level significance was established using the Kolmogorov-Smirnov test (K-S test) using the ChemRICH method, Tanimoto coefficients were calculated to asses intra-set chemical similarity using Metabox, and the frequency of occurrence in the published literature and relevant databases considered (KEGG, ChEBI, Human Metabolome

Database, Human Breathomics Database and microbial VOC database). Chemical similarity is of interest because compounds derived from similar pathways may also share common structural features or chemical groups. This combined data-driven and chemistry-driven approach has been shown to improve enrichment analysis and allowed further interpretation core findings herein (FIG. 11).

### Supplementary Information (SI)

Probability distributions of breath features (biomarkers):

The features in the GCxGC peak table fell into 3 broad categories: (1) constant features (all samples had a value of zero), (2) features that contained a mixture of zero and non-zero values, and (3) features that contained all non-zero values. The zero values have arisen owing to the measurement being below the instrument's lower limit of detection. Constant features were removed prior to fitting the main model.

Graphical distribution of the final 101 features (biomarkers), mainly falling into type 2 and 3 categories is illustrated in (FIG. 8). For certain features the spike in the 0 values can be clearly seen. Based on these observations a reasonable choice for a theoretical model for the probability distribution of a feature from a GCxGC-MS peak table might be the Zero Modified Log Normal distribution.

Mitigating the adverse impact of batch effects in biomarker pattern detection

Batch effect is a common issue in omics data analysis. The existence of batch effects makes it challenging to compare data collected and analysed at different processing times (FIGS. 9 & 10).

The following factors were investigated as possible contributing batch variation factors:

I. Batch_ID—date of sample collection:
(1) Batch 1—August 2017-October 2017
(2) Batch 2—November 2017-March 2018
(3) Batch 3—April 2018-December 2018
   II. Operator: (N: 1-6)—indicating members of the study team operating the RECIVA over the entire course of the sampling program
   III. Time of the day sample was collected (circadian rhythm):
(1) 1=between 9-11am
(2) 2=between 11am-1pm
(3) 3=between 1-3pm
(4) 4=between 3-5pm
   IV. Time sample stored wet
(1) 1=0-2 days
(2) 2=2-5 days
(3) 3 =5-10 days
(4) 4 =10-20 days
(5) 5 =20-42 days
(6) 6 =over 42 days
   V. Time stored dry (following dry purging)
(1) 1=0-2 days
(2) 2=2-5 days
(3) 3=5-10 days
(4) 4=10-20 days
(5) 5=20-42 days
(6) 6=over 42 days
   VI. Volume of breath collected (over 80% threshold):
(1) 1=100%
(2) 2=90-99%
(3) 3=80-89%

FIG. 9 is a visualization of the GCxGC-MS peak table comprising all 805 features using t Stochastic Nearest Neighbor Embedding (tSNE). Clustering due to ‘date of collection’ was seen (top left plot). No obvious clustering seemed to be present for the remaining factors. The effect collection date was adjusted for by applying Parametric Empirical Bayesian Adjustment (PEBA). The ComBat function from the SVA package for Bioconductor was used to perform PEBA. The results of this adjustment are shown in (FIG. 10). It can be seen that the clustering due to collection date is no longer apparent. The batch effect adjusted peak table was used in all subsequent feature selection models.

### Model Accuracy

The overall classification accuracy for the statistical model using all five biomarker scores from the final set of 101 exhaled breath features was assessed by comparing the balanced accuracy of model trained using the true class labels versus the balanced accuracy of the same model tested using randomly shuffled class labels. This process was repeated 1000 times. The overall classification accuracy using all five biomarker scores was 0.722, 95% CI (0.6653-0.774) and the results demonstrated in FIG. 14.

Chemical speciation of identified breath biomarkers

In order to confirm to the chemical identity of the concatenated list of 101 exhaled breath peaks, a standard reference compounds, where available, were purchased and analysed. This included a C8-C20 saturated alkanes certified reference material (Sigma Aldrich, Dorset, UK), an aromatics calibration standard (NJDEP EPH 10/08 Rev.2, Thames Restek, Saunderton, UK), a multi-component indoor air standard (Sigma Aldrich, Dorset, UK), two terpene reference mixtures (Spex Centriprep, Emerald Scientific, San Luis Obispo, US), and individual standards from Sigma Aldrich (Merck Life Sciences), Greyhound Chromatography, Scientific Lab Supplies, Alfa Chemicals and Santa Cruz Biotechnology.

FIG. 16 lists the chemical assignment of the selected predictive markers from the regression model detailing chemical name, CAS registry number, KEGG, Human Metabolome Database and ChEBI identifiers and MSI-compliant metabolite identification level, concentration range and fold change (expressed as log2) between acute and control groups, and compound contribution towards disease-specific biomarker risk scores (†adjusted p-value<0.05).

Sample size estimation

In the study protocol the aim was to recruit 550 subjects, had 550 subjects been recruited then we would be powered to identify sensitive biomarkers (≥80%) of acute breathlessness with a maximum marginal error in the estimate for sensitivity not exceeding 5% with 95% confidence. Similarly, we are powered to identify specific biomarkers (>80%) of acute breathlessness with a maximum marginal error in the estimate for specificity not exceeding 5% with 80% confidence, however we have achieved a total sample size of n=277.

Based on a total sample size of n=277 post hoc sample size calculations were performed using a sensitivity of 70% and 80% with + (10%, 15% and 20% precision) for obtaining a biomarker capable of ‘ruling out’ an acute disease class. The same targets were applied to specificity. Calculations were performed for using a 95% confidence level.

It was assumed an 80% acute disease prevalence for recruitment and 1:5 patients recruited were non-breathless healthy controls (Table 2). It was acknowledged that the assumption of an 80% acute disease prevalence places a limitation on the validity of the sample size calculations, however the estimate of 80% prevalence is not unreasonable based on clinical expectation.

TABLE 2
demonstrates that the sample sizes in discovery
(n=139) and replication (n=138) are sufficient to identify
sensitive and specific biomarkers (≥ 70%) of acute
breathlessness with a maximum marginal error in the
estimate for sensitivity not exceeding 20% (95%
confidence). Similarly, from Table 2 the sample sizes
in discovery and replication are sufficient
to identify sensitive and specific biomarkers
(≥ 80%) of acute breathlessness with a
maximum marginal error in the estimate for
specificity not exceeding 15% (95% confidence).

| | | | Prevalence | |
|---|---|---|---|---|
| | Confi- | | of Acute | Sample |
| | dence | Marginal | Breath- | Size |
| | Level | Error | lessness | Required |
| Sensitivity | 95% | 10% | 80% | 100 |
| (70%) | | | | |
| Specificity | 95% | 10% | 80% | 403 |
| (70%) | | | | |
| Sensitivity | 95% | 15% | 80% | 45 |
| (70%) | | | | |
| Specificity | 95% | 15% | 80% | 180 |
| (70%) | | | | |
| Sensitivity | 95% | 20% | 80% | 25 |
| (70%) | | | | |
| Specificity | 95% | 20% | 80% | 100 |
| (70%) | | | | |
| Sensitivity | 95% | 10% | 80% | 77 |
| (80% | | | | |
| Specificity | 95% | 10% | 80% | 307 |
| (80%) | | | | |
| Sensitivity | 95% | 15% | 80% | 34 |
| (80%) | | | | |
| Specificity | 95% | 15% | 80% | 137 |
| (80% | | | | |
| Sensitivity | 95% | 20% | 80% | 19 |
| (80%) | | | | |
| Specificity | 95% | 20% | 80% | 77 |
| (80%) | | | | |

Co expression and feature enrichment analysis
Graph construction and Cluster Analysis

Subjects from both the Discovery and Replication sets were combined into a data matrix [symbol] _{D} comprising the 101 features that were obtained from previous regression analysis, with healthy subjects excluded. The Spearman rank correlation matrix was calculated for the data matrix [symbol] _{D}.

A scale free graph g was constructed by generating the adjacency matrix [symbol] _{Adj}=|Ĉ|^{β}.

Where Ĉ is the sample correlation matrix of [symbol] _{D}, and β≥1.

The pickSoftThreshold function from the WGCNA package in R was used to estimateβ. The igraph package in R was used to construct g using [symbol] _{Adj}, g is a weighted and unsigned graph. The graph g will be referred to as the “correlation graph”.

Louvain clustering was then performed on the correlation graph and 8 feature sets were obtained.

The 8 feature sets obtained from Louvain clustering on correlation graph were used in an enrichment analysis. Instead of considering individual features and how they might distinguish different disease groups, sets of features are considered, the idea being that features in combination may have better discriminatory capability. The bioconductor (version 3.12) packages GSVA and limma were used to perform enrichment analysis. Feature set 3 was found to be enriched in Asthma and HF, feature set 5 was found to be enriched in HF alone, see Tables 3-6. The enriched feature sets 3and 5 did not demonstrate improved diagnostic accuracy over the scores obtained from regression analysis.

TABLE 3
Demonstrates the results of the enrichment analysis performed in the asthma
group using the 8 feature sets obtained from the Louvain clustering on the correlation
graph (FIG. S9)

| | logFC | AveExpr | t | P. Value | adj.P.Val | B |
|---|---|---|---|---|---|---|
| Set | 0.133890616 | 0.004263125 | 2.879346419 | 0.004319299 | 0.034554392 | −2.142608513 |
| 3 | | | | | | |
| Set | −0.120196744 | 0.011684816 | −1.854952871 | 0.064743701 | 0.258974804 | −4.369194843 |
| 1 | | | | | | |
| Set | 0.061793373 | 0.015703055 | 1.157435072 | 0.248165085 | 0.544802368 | −5.347558772 |
| 7 | | | | | | |
| Set | −0.060241661 | −0.027387588 | −0.996694751 | 0.31984657 | 0.544802368 | −5.50959215 |
| 5 | | | | | | |
| Set | −0.044261401 | 0.018231244 | −0.830155612 | 0.407218311 | 0.544802368 | −5.652155219 |
| 6 | | | | | | |
| Set | 0.042555754 | 0.007308014 | 0.827706348 | 0.408601776 | 0.544802368 | −5.65405913 |
| 2 | | | | | | |
| Set | −0.035370385 | 0.012747522 | −0.700755726 | 0.484086974 | 0.553242256 | −5.74507753 |
| 4 | | | | | | |
| Set | 0.007185736 | 0.005059851 | 0.128594967 | 0.897778335 | 0.897778335 | −5.967968995 |
| 8 | | | | | | |

TABLE 4
Feature enrichment in COPD using 8 features sets obtained by Louvain
clustering on the correlation graph.

| | logFC | AveExpr | t | P. Value | adj.P.Val | B |
|---|---|---|---|---|---|---|
| Set3 | −0.090944102 | 0.004263125 | −1.847462511 | 0.065824888 | 0.31930245 | −4.015728578 |
| Set5 | −0.101063794 | −0.027387588 | −1.579494626 | 0.115447869 | 0.31930245 | −4.359177814 |
| Set6 | 0.081933094 | 0.018231244 | 1.451613602 | 0.147823971 | 0.31930245 | −4.504601143 |
| Set1 | 0.096742783 | 0.011684816 | 1.410314835 | 0.159651225 | 0.31930245 | −4.548997592 |
| Set4 | 0.058154903 | 0.012747522 | 1.08835486 | 0.277454107 | 0.443926572 | −4.851845137 |
| Set7 | 0.044836139 | 0.015703055 | 0.793305124 | 0.428329034 | 0.489518896 | −5.061530212 |
| Set8 | 0.002536665 | 0.005059851 | 0.042881813 | 0.965828915 | 0.965828915 | −5.299201629 |

TABLE 5
feature enrichment in heart failure using 8 features sets obtained by
Louvain clustering on the correlation graph.

| | logFC | AveExpr | t | P.Value | adj.P.Val | B |
|---|---|---|---|---|---|---|
| Set3 | −0.16062091 | 0.004263125 | 2.841944019 | 0.004842214 | 0.032792407 | −2.229765975 |
| Set5 | 0.195734539 | −0.027387588 | 2.664418025 | 0.008198102 | 0.032792407 | −2.6741005 |
| Set2 | 0.147456886 | 0.007308014 | 2.359677827 | 0.019036076 | 0.050762869 | −3.37356668 |
| Set1 | 0.087558967 | 0.011684816 | 1.111758525 | 0.267276909 | 0.534553819 | −5.37643629 |
| Set6 | 0.054112112 | 0.018231244 | −0.835023125 | 0.404477256 | 0.64716361 | −5.628009236 |
| Set7 | −0.040272554 | 0.015703055 | −0.620631141 | 0.535390206 | 0.711290217 | −5.773994793 |
| Set4 | 0.028289272 | 0.012747522 | 0.461124606 | 0.645097706 | 0.711290217 | −5.85478987 |
| Set8 | −0.025165368 | 0.005059851 | −0.370531932 | 0.711290217 | 0.711290217 | −5.890086764 |

TABLE 6
feature enrichment in Pneumonia using 8 features sets obtained by Louvain
clustering on the correlation graph.

| | logFC | AveExpr | t | P.Value | adj.P.Val | B |
|---|---|---|---|---|---|---|
| Set2 | 0.092675555 | 0.007308014 | 1.658089062 | 0.098514794 | 0.350018518 | −4.257598246 |
| Set3 | 0.083374576 | 0.004263125 | 1.6493092 | 0.100301314 | 0.350018518 | −4.268325935 |
| Set6 | 0.082784378 | 0.018231244 | 1.428260455 | 0.154426336 | 0.350018518 | −4.520076707 |
| Set5 | −0.08936284 | −0.027387588 | −1.36002492 | 0.175009259 | 0.350018518 | −4.590632855 |
| Set8 | 0.029388432 | 0.005059851 | 0.483786536 | 0.628947737 | 0.86559581 | −5.192394684 |
| Set7 | −0.024708996 | 0.015703055 | −0.425730243 | 0.670659348 | 0.86559581 | −5.212141706 |
| Set1 | 0.017148026 | 0.011684816 | 0.243432731 | 0.807863636 | 0.86559581 | −5.257780971 |
| Set4 | 0.009296591 | 0.012747522 | 0.169424129 | 0.86559581 | 0.86559581 | −5.269216827 |

### Example 1—Overview

Exhaled breath from 277 participants, recruited from acutely breathless hospitalised patients and matched healthy controls, was sampled and analysed to identify dysregulation of metabolic classes in cardio-respiratory disease and investigate whether exhaled VOC profiles could predict acute cardio-respiratory exacerbations despite diagnostic uncertainty, and thus have a potential role in phenotyping acute cardio-respiratory breathlessness.

Participants' mean (SD) age was 60.8 ±(16.8) years, 51% were males, 30 patients required supplemental oxygen on admission and the mean admission modified early warning score (mEWS−2 score) was 2. The cohort was made up of patients presenting with the following exacerbation subtypes; acute severe asthma (n=65), acute severe COPD (n=58), acute severe heart failure (n=44), community acquired pneumonia (n=55), and healthy volunteers (n=55), recruited between May 2017 and December 2018 (FIG. 6). Participants' demographic and clinical characteristics are summarised in Table 7. Breath samples were collected using a ReCIVA© device, adopting a standardised sampling and gated protocol that enriches alveolar volatiles, and analysed using thermal desorption (TD) coupled to comprehensive two-dimensional gas chromatography (GCxGC) with dual flame ionisation detection (FID) and mass spectrometry (MS) (FIG. 1 and Methods).

TABLE 7
Demographics and clinical characteristics of study participants.

| | Total | Healthy | Acute | Acute | | Heart | p |
|---|---|---|---|---|---|---|---|
| | no | controls | asthma | COPD | Pneumonia | failure | value |
| Total no of | 277 | 55 | 65 | 58 | 55 | 44 | |
| participants (n=) | | | | | | | |

Demographics

| | | | | | | | |
|---|---|---|---|---|---|---|---|
| Age *, years | 60.8± | 63.05± | 44.3± | 69.82± | 60.67± | 70.72± | 124 |
| | (16.8) | (11.78) | (17.93) | (8.16) | (16.50) | (11.04) | |
| Gender | 143 | 26 | 25 | 33 | 27 | 32 | |
| Male (n=) (%) | (51%) | (47%) | (38%) | (56%) | (49%) | (72%) | .008 ¥ |
| Body Mass Index | 29.5± | 28.2± | 31.5± | 27.5± | 29.2± | 31.5± | .767 |
| (BMI)*^{a} | (7.3) | (4.5) | (9.0) | (7.7) | (6.9) | (6.5) | |
| Smoking | | | | | | | |
| Current smoker | 53 | 4 | 13 | 21 | 11 | 4 | .001 ¥ |
| (n=) (%) | (19%) | (7%) | (20%) | (36%) | (20%) | (9%) | |

Vital signs

| | | | | | | | |
|---|---|---|---|---|---|---|---|
| Temperature | 36.7± | 36.1± | 36.8± | 36.7± | 37.1± | 36.5± | .000 |
| (Celsius)* | (0.6) | (0.4) | (0.5) | (0.5) | (0.7) | (0.3) | |
| Heart rate | 87.2± | 68.1± | 99.6± | 92.9± | 90.3± | 81.3± | |
| (beats/min)* | (18.5) | (9.54) | (17.2) | (15.6) | (15.4) | (15.6) | .005 |
| Respiratory rate | 18.9± | 13.0± | 20.5± | 21± | 20.4± | 19.1± | .000 |
| (breaths/min)* | (4.2) | (1.8) | (3.4) | (2.5) | (4.6) | (1.8) | |
| Oxygen | 95.8± | 97.7± | 96.1± | 94.0± | 94.5± | 96.5± | .001 |
| saturations (%)* | (3.0) | (1.3) | (2.5) | (2.9) | (0.5) | (1.9) | |
| Systolic Blood | 131.5± | 134± | 133± | 133± | 126± | 128± | .515 |
| Pressure | (19.2) | (15.7) | (17.7) | (20.5) | (19.4) | (22.2) | |
| (mmHg)* | | | | | | | |
| Total mEWS-2 | 1 | 0 | 2 | 3 | 2 | 1 | .000 |
| score \hat{ }^{b} | (0-3) | (0-1) | (1-3.5) | (1-5) | (1-3) | (0-2) | |

Symptoms
assessment

| | | | | | | | |
|---|---|---|---|---|---|---|---|
| Breathlessness | 58.1± | 6.2± | 76.6± | 71.6± | 67.8± | 67.9± | .000** |
| VAS score | (31.6) | (9.3) | (14.2) | (19.2) | (22.1) | (20.0) | |
| (mm)*^{c} | | | | | | | |
| Cough VAS score | 43.3± | 8.7± | 64.5± | 57.8± | 53.6± | 24.3± | .000** |
| (mm) *^{c} | (33.2) | (14.3) | (26.7) | 27.0) | (30.6) | (25.2) | |
| Wheeze VAS | 41.8± | 3.4± | 66.2± | 60.3± | 45.1± | 28.1± | .000** |
| score (mm) *^{c} | (34.9) | (6.4) | (24.5) | (29.0) | (34.8) | (28.6) | |

eMRC^{d} score
(n=) (%)

| | | | | | | |
|---|---|---|---|---|---|---|
| 1 | 17 | 1 | 8 | 7 | 1 | .000¥ |
| | (6%) | (1.5%) | (13%) | (12%) | (2%) | |
| 2 | 6 | 0 | 0 | 5 | 1 | .000¥ |
| | (2%) | (0%) | (0%) | (9%) | (2%) | |
| 3 | 15 | 6 | 0 | 7 | 2 | .000¥ |
| | (5%) | (10%) | (0%) | (12%) | (4.5%) | |
| 4 | 50 | 16 | 11 | 6 | 17 | |
| | (18%) | (25%) | (19%) | (11%) | (38.5%) | .000¥ |
| 5a | 112 | 38 | 32 | 22 | 20 | |
| | (40%) | (51%) | (55%) | (41%) | (46%) | .000¥ |
| 5b | 21 | 3 | 7 | 8 | 3 | .000¥ |
| | (7%) | (4.5%) | (13%) | (15%) | (7%) | |

Exposure to
antibiotics and
steroids within 2
weeks of hospital
admission

| | | | | | | | |
|---|---|---|---|---|---|---|---|
| Antibiotics (n=) | 61 | n = 0 | n = 24 | n = 23 | n = 10 | n = 4 | .002¥ |
| (%) | | (0%) | (36.9%) | (39.6%) | (18.2%) | (9.0%) | |
| Steroids (n=) (%) | 57 | n = 0 | n = 28 | n = 24 | n = 3 | n = 2 | .000¥ |
| | | (0%) | (43.0%) | (41.3%) | (5.4%) | (4.5%) | |

Morbidity and
mortality
measures

| | | | | | | | |
|---|---|---|---|---|---|---|---|
| Length of | 3 | | 2.0 | 4.0 | 4.0 | 7.0 | .000** |
| hospital stay | (2-6) | | (1.0-3.0) | (2.0-6.0) | (2.0-5.0) | (4.0-11) | |
| (days) \hat{ } | | | | | | | |
| 30-60 days | | | | | | | |
| hospital | 29 | | 7 | 9 | 6 | 7 | .461¥ |
| readmission (n=) | | | | | | | |
| 1 year all-cause | 12 | 0 | 1 | 5 | 1 | 5 | .078¥ |
| mortality | | | | | | | |

Laboratory
parameters

| | | | | | | | |
|---|---|---|---|---|---|---|---|
| C-reactive protein | 11 | 5 | 10.0 | 12.0 | 108.0 | 11.0 | .000** |
| (CRP) (mg/L)\hat{ } | (5.0-34.2) | (5-5) | (5.0-23.0) | (5.0-20.7) | (53.5-245.3) | (5.0-22.0) | |
| Blood Eosinophil | 0.13 | 0.17 | 0.18 | 0.13 | 0.08 | 0.13 | .000** |
| count 10^{Λ}9/L\hat{ } | (0.06-0.24) | (0.09-0.24) | (0.06-0.42) | (0.06-0.24) | (0.04-0.14) | (0.08-0.23) | |
| Troponin T | 3.3 | 2.05 | 1.55 | 3.75 | 4.3 | 20.2 | .000** |
| (ng/l)\hat{ } | (1.0-11.4) | (1.0-2.7) | (1.0-3.4) | (2.6-10.9) | (2.18-11.3) | (13.4-59.6) | |
| Brain natriuretic | 40.5 | 28.40 | 20.4 | 56.3 | 56.3 | 611.8 | .000** |
| peptide (BNP) | (20.6-98.9) | (17.60-39.88) | (12.1-40.0) | (24.3-95.0) | (27.4-132.1) | (172.1-1259.1) | |
| (ng/l)\hat{ } | | | | | | | |

Questionnaires

| | | | | | |
|---|---|---|---|---|---|
| Asthma Quality | 65 | 117.3± | | | |
| of Life | | (37.3) | | | |
| Questionnaire | | | | | |
| (AQLQ) total* | | | | | |
| COPD | | | | | |
| Assessment test | 58 | | 26.7± | | |
| (CAT) * | | | (7.3) | | |
| COPD Decaf | 58 | | 1.7 | | |
| score * | | | (0.8) | | |
| CURB65 score\hat{ } | 55 | | | 2 | |
| | | | | (1-3) | |
| NYHA score\hat{ } | 44 | | | | 2 |
| | | | | | (1-3) |

Continuous variables are presented as mean ± standard deviation. Categorical variables are presented as numbers (%).
^{a}The body mass index (BMI) is the weight in kilograms divided by the square of the height in meters.
^{b}Modified Early warning score-2 (MEWS-2) is a guide widely used by medical services to determine the degree of illness of a patient based on their vital signs including respiratory rate, oxygen saturations, temperature, blood pressure, and heart rate. Vital signs collected at the point of admission for acute disease groups.
^{c}Participants were asked to determine their degree of breathlessness, cough and wheeze on a 100 mm visual analogue scale (VAS) on admission. Higher scores indicate worse symptoms.
^{d}Extended Medical research Council (eMRC) scale is a validated measure of perceived respiratory disability, scored from 1 to 5b. Higher scores indicate worse disability.
*Data is expressed as mean (SD) or n (%) ± (SD),
\hat{ }Data expressed as median (IQ range),
**Kruskal-Wallis test comparing non-parametric data,
¥Pearson Chi Squared and Fisher's Exact test.
ANOVA was used to assess the differences between groups for normally distributed continuous variables and kruskal-Wallis for non-parametric continuous variables. Pearson chi-squared and Fisher's exact were used to assess the differences in categorical variables. The results were considered statistically significant at p-values < 0.05.

### Example 2—Unbiased Discovery Using Topological Data Analysis Identifies Breath Markers of Acute Disease

To achieve an unbiased discovery of exhaled VOCs predictive of the acute disease groups, patients were block randomised post-hoc into a discovery cohort of 139participants (acute asthma n=33, acute COPD n=29, acute heart failure n=22,community acquired pneumonia n=28, healthy volunteers n=27), and a replication cohort of 138 participants (acute asthma n=32, acute COPD n=29, acute heart failure n=22,community acquired pneumonia n=27,healthy volunteers n=28). Randomisation allowed internal replication of diagnostic breath biomarkers, whilst adjusting for relevant confounders. Details of the randomisation and further clinical characteristics of the cohorts can be found in Methods and Tables 1 and 8.Chemometric analysis and quantification of VOCs was performed blinded to clinical diagnosis by two analytical chemists (MW and RC), with bio-statistical analyses linking subject identifier to chemometric biomarkers performed following data lock by an independent statistician (MR).

805 unique chromatographic features (peaks) were detected across the breath sample set using TD-GCxGC-FID/MS. Topological data analysis (TDA) applied to these 805chromatographic features, yielded topologically distinct networks that distinguished underlying causes of acute breathlessness whilst anchoring to corresponding blood-based biomarkers in both the discovery and replication cohorts (FIG. 2). Specifically, healthy volunteers and patients with acute heart failure formed distinct topological groupings in both discovery and replication populations, whilst respiratory admissions due to acute asthma, acute COPD and pneumonia formed a topological continuum albeit within distinct regions of a single network in the replication cohort with similar findings in the discovery cohort, with the except of acute asthma forming a distinct grouping.

TABLE 8
Baseline demographics and clinical characteristics of the discovery and
replication cohorts. VAS: Visual Analogue Scale (100mm), participants were asked to rate
their breathlessness, cough and wheeze on a 100mm VAS on admission. ANOVA was used
to assess the differences between groups for normally distributed continuous variables
and kruskal-Wallis for non-parametric continuous variables. Pearson chi-squared and
Fisher's exact were used to assess the differences in categorical variables. The results
were considered statistically significant at p-values <0.05.

| | Discovery | Replication | p value |
|---|---|---|---|
| Total number (n=) | 139 | 138 | |
| Acute asthma (n=) | 33 | 32 | |
| Acute COPD (n=) | 29 | 29 | |
| Pneumonia (n=) | 28 | 27 | |
| Heart failure (n=) | 22 | 22 | |
| Healthy volunteers (n=) | 27 | 28 | |
| Demographics | | | |
| Age (years) mean ± (SD) | 60.6 ± (16.9) | 61.0 ± (16.8) | .846 |
| Gender Male (n=) (%) | 65 (46%) | 78 (56%) | .104¥ |
| Height (meters)* | 1.66 ± (0.13) | 1.68 ± (0.16) | .215 |
| Weight (kilograms)* | 82.5 ± (21.1) | 85.7 ± (25.6) | .260 |
| Body Mass Index (BMI)* | 29.5 ± (6.7) | 29.6 ± (7.9) | .896 |
| Breathlessness | | | |
| Breathlessness VAS score (mm)* | 56.1 ± (32.3) | 60.2 ± (30.7) | 0.292 |
| V1 cough VAS score (mm)* | 41.6 ± (33.3) | 44.5 ± (33.2) | 0.479 |
| V1 wheeze VAS score (mm)* | 40.65 ± (35.1) | 43.1 ± (34.8) | 0.558 |
| Laboratory parameters | | | |
| C-Reactive Protein (mg/dl) | 10.0 (1.0-449.0) | 12.0 (1.0-321.0) | 0.740 |
| Blood eosinophil count 10\hat{?}9/L | 0.13 (0.01-1.9) | 0.13 (0.01-2.15) | 0.825 |
| Troponin T (ng/l) | 3.4 (1.0-1658.4) | 3.15 (1.0-810.1) | 0.565 |
| BNP (ng/l) | 40.1 (1.0-1576.0) | 42.6 (1.0-2631.9) | 0.780 |

* Data is expressed as mean (SD) or n (%) ± (SD).

### Example 3—Biomarker Profiling and Risk Scores

In order to create a concatenated list of exhaled breath biomarkers suitable for diagnostic application, a threshold of 80% feature-presence per patient group was applied, below which features were removed (FIG. 7). This approach was further supported by the unique distribution properties of breath biomarkers (FIG. 8) and to enable the generation of patient specific multi VOC biomarker risk scores. Further filtering steps using Least Absolute Shrinkage and Selection Operator (LASSO) and Elastic Net regression methods, followed by removal of 38 peaks that were considered to be chemical and material artefacts (e.g. siloxanes), and generated a final panel of 101 exhaled breath volatiles (FIG. 7). Therefore, the analysis plan permitted the identification of a rich and chemically diverse response in the VOC profile as opposed to only a handful of individual VOC markers and afforded the generation of biomarker risk scores. The data was examined for batch effects and was adjusted accordingly. Batch effects detected related to major instrument maintenance events (which occurred twice creating three groups, see Supplementary Information section on batch adjustment). No significant contributions were observed based on the ReCIVA device used, operator, time of day, or volume of breath sample collected, most likely nullified by the simultaneous and consecutive recruitment across all cohorts throughout the study to reduce potential biases (FIG. 9-10). The value of the generated VOC biomarker risk score was found to be significantly higher in acute cardio-respiratory patients compared to healthy volunteers (FIG. 3a). For the discovery cohort (n=139), the VOC biomarker risk score was able to effectively differentiate participants with acute cardio-respiratory exacerbations from age-matched healthy controls with an area under the curve (AUC) of 1.00 (1.00-1.00) p<0.0001,sensitivity 1.00 (1.00-1.00), specificity (1.00-1.00), positive predictive value (PPV) 1.00 (1.00-1.00), negative predictive value (NPV) (1.00-1.00). For the replication cohort (n=138), the same VOC biomarker risk score differentiated participants with acute disease from healthy controls with AUC 0.89 (0.82-0.95) p<0.0001, sensitivity 0.79 (0.71-0.86), specificity AUC 0.85 (0.72-0.98), PPV of 0.95(0.91-0.99), NPV of 0.51 (0.36-0.65) (FIG. 3b).

Following a clinical adjudication process (Methods), each patient was assigned a degree of clinical diagnostic uncertainty using a 100 mm visual analogue scale (VAS) at the point of clinical triage (FIG. 3c). Diagnostic uncertainty was defined as patients with values higher than or equal to the upper quartile of 20 mm on the VAS. The acute disease VOC biomarker risk score was able to identify acute disease with an AUC 0.96 (0.92-0.99) p<0.0001, sensitivity 0.90 (0.82-0.97), specificity 0.92 (0.85- 0.99), PPV 0.93 (0.86-0.99), NPV 0.89 (0.81-0.97) (FIG. 3d).

Further comparative ROC analysis was performed to assess the diagnostic accuracy of asthma biomarker score against predominantly infection-driven respiratory illnesses (Pneumonia and COPD) in the pooled cohort curve AUC: 0.70 (0.62-0.78) p<0.0001,sensitivity 0.72 (0.64-0.83), specificity 0.64 (0.55-0.73), PPV 0.54 (0.43-0.64), NPV 0.80 (0.72-0.88). ROC analysis was performed to assess the diagnostic value of heart failure biomarker score against other acute disease groups AUC: 0.78 (0.70-0.86) p<0.0001, sensitivity 0.77 (0.64-0.89), specificity 0.71 (0.64-0.78), PPV 0.40 (0.29- 0.50), NPV 0.92 (0.88-0.97) (FIG. 15).

### Example 4—Correlation of Exhaled Breath Biomarker Scores with Blood-Based Biomarkers and Admission Observations

As previously described, VOC biomarker risk scores were generated for each of the acute disease subgroups and healthy subjects without cardio-respiratory breathlessness. There was a weak, but statistically significant positive correlation, in the combined discovery and replication cohorts (n=277), between the VOC scores for pneumonia and CRP (n=277, r=0.33, p<0.0001), acute heart failure and BNP (n=277,r=0.33, p<0.0001), in addition to a significant negative correlation between the healthy-state VOC score and CRP and BNP (n=277, r=−0.15, p<0.0001,and −0.21,p<0.0001 respectively) (FIG. 4a).

Interestingly, significant correlations were also identified between the acute disease VOC score and vital observations carried out during triage (FIG. 4b).

### Example 5—Chemical Classification of Predictive Markers in Disease Groups

Chemical identification of the 101 biomarker panel involved comparison with an authentic reference compound in accordance with the Metabolomics Standard Initiative (MSI) Level 1 criteria for metabolite identification (FIG. 16).

The most common chemical classes associated with acute breathlessness in this study included straight-chain and methyl-branched hydrocarbons (30%), ketones (10%), aldehydes (8%) and terpenes (13%), followed by sulphur-containing VOCs (7%), alcohols (6%), aromatics (5%), esters (3%), nitrogen-containing VOCs (3%), ethers (2%), halogen-compounds (1%), and an assortment of other less prevalent and less relevant classes such as acrylates (12%) (FIG. 16).

### Example 6—Metabolite Set Enrichment and Chemical Similarity Analysis

Unlike functional indications, which are reliant on mapping metabolites with known well-annotated metabolic pathways, metabolic changes indicative of response can be derived independently. To derive clues of responsive indication, the panel of 101 features was assessed for covarying clusters i.e. metabolite sets (FIG. 5A, and FIG. 11).

Overall twenty metabolite sets were identified, eleven of which were enriched during acute cardio-respiratory exacerbations. The seven metabolite sets that were upregulated consisted of predominantly acyclic and branched hydrocarbons (sets 3, 5, 7 and 9 in FIG. 11). The results from the analysis herein demonstrate significantly enriched, co-expression of hydrocarbons with high chemical similarity providing primary evidence of exhaled VOCs indicative of disease response measured in vivo. This is clearly seen in (FIG. 5a), with the metabolite sets (inner tree) labelled by broader chemical classifications (outer ring): C₅₋₇, C₈₋₁₀ and C₁₁₋₁₆ form clusters based on carbon number also exhibiting the highest change during acute exacerbation.

### Example 7—Diagnostic Accuracy of Breath Biomarker Scores in Cardio-Respiratory Disease Subgroups

A multinomial regression model using elastic net regularization was fitted to the matrix of 101 breath biomarkers with the 10-fold cross validation repeated 100 times. Linear combinations of the most stable features from the multinomial regression model fitted to the 101 biomarkers formed a set of scores for predicting probability of belonging to the different disease groups (acute Asthma, acute COPD, pneumonia, heart failure or healthy volunteers). The median values of the exhaled breath VOC scores and their distribution across disease subgroups are detailed in FIG. 12.

For the pooled cohort (n−277) the overall classification accuracy using all five biomarker scores was 0.722, 95% CI (0.6653-0.774) (FIG. 14). The balanced accuracy for acute asthma was 0.8274,for acute COPD 0.7751,for heart failure 0.7967,for community acquired pneumonia 0.7935,and for healthy controls was 0.9274.

Discussion In this pragmatic, acute-care study, the validity of breath biomarker profiling in high-acuity patients presenting with acute cardio-respiratory breathlessness was evaluated. Using GCxGC-MS, the inventors observed that robust and validated sampling of alveolar breath coupled with GCxGC-MS biomarker characterisation demonstrated high diagnostic accuracy for acute cardio-respiratory exacerbations. Putative biomarker risk scores from subsets of breath VOC biomarkers that classify cardio-respiratory exacerbation subtypes and warrant validation in replication studies have also been identified. Furthermore, several classes of VOCS that are highly correlated and selectively enriched or supressed in acute disease (including subgroups), compared to health, providing potential insights into broad dysregulation of the metabolome in acute cardio-respiratory exacerbations have been identified.

This study is the first to attempt to characterise exhaled breath VOCs in a large cohort with severe cardio-respiratory exacerbations and the results position this study as a proof-of-concept for the use of breathomics in acute clinical settings.

The analytical methods described herein were underpinned by robust biomarker development protocols using TD-GCxGC-FID/MS, integral to the standardisation and integration of breath analysis in large translational studies. Several potential confounders including batch variation, were addressed in detail (SI). Furthermore, biomarker quantification of the 101 VOC modelled followed the recommendations of the Metabolomics Standard Initiative (MSI) with 58 compounds identified against pure and traceable standards (level I), 21 putative identities based on mass spectral and retention index library matches (level 2) and 22 classified on mass spectral data

FIG. 16. Markers that appeared to localise to individual cardio-respiratory conditions could be readily visualised (FIG. 5).

The identification of hydrocarbons and carbonyls as the major chemical classes was consistent with current mechanistic understanding, postulated as chemical endpoints of lipid peroxidation, a result of oxidative stress during inflammation. Aldehydes such as nonanal, decanal and hexanal were predictive for asthma, ketones included 2-pentanone (asthma), cyclohexanone (pneumonia) and 2,3-butanedione (COPD). Individual hydrocarbons such as 2,4-and 2,2-dimethylpentane: 2-methylbutane, 4-methyldecane, 5-methylnonane and isoprene are predictive for pneumonia and heart failure. Sulphur-containing VOCs, such as 3-methylthiophene, allyl methyl sulphide and carbonyl sulphide (found to be predictive of COPD) are associated with bacterial metabolism, postulated to originate from the gut and on occasions as a result of radiation injury. 2,3-butanedione is also predictive of COPD.

Not all the compounds were considered to be endogenous VOCs, with 27 attributed to contamination from personal care products such as cosmetics FIG. 16. Eleven of the features predictive of the control group were assigned as either fragrances (e.g. alpha isomethyl ionone) or waxy long-chain chemicals used in cosmetics as emollients and surfactants (e.g. stearyl vinyl ether and isopropyl myristate). These were likely captured in the breath sample because of the proximity of the sorbent tubes to the patients face.

Co-expression and enrichment analysis of the Louvain clusters on the correlation graph (Feature enrichment analysis section-Tables 3-6), revealed a set of highly correlated metabolites significantly enriched in specific disease groups. Comparison of the Louvain clusters with the metabolite sets identified using the method previously described, demonstrated strong overlap (FIG. 5A and 5B). The metabolites enriched in heart failure were a cluster of highly correlated C₅₋₇ hydrocarbons and C₃₋₅ carbonyls with high chemical similarity (based on Tanimoto coefficients as determined in Methods and FIG. 11. The cluster included 2,4-and 2,2-dimethylpentan; 2-methylbutane, 2-methyl-1,3-butadiene (isoprene), 3-methylpentane, hexane and cyclohexane.

The analysis also revealed a separate set of highly correlated aldehydes (nonanal, decanal, undecanal, and a methyldecanal isomer), lower in acute exacerbations of asthma compared with acute exacerbations of COPD and pneumonia. Depletion of VOCs during in vitro experiments has been reported as a consequence of metabolic activity by immune cells, but the association herein is tentative and should be interpreted with caution due to the correlation between inhaled air and exhaled air concentrations of these compounds (median Spearman rank=0.60), also previously observed.

In conclusion, the inventors have conducted an acute care volatile breath biomarker study using robust clinical and analytical technology and have identified high diagnostic sensitivity and specificity of biomarkers in acute cardio-respiratory disease, alongside robust biomarker identification and mechanistic association warranting further metabolomic phenotyping approached in acute cardio-respiratory exacerbations.

1. (canceled)

2. A method of treating a cardiorespiratory disease in a subject, the method comprising:
detecting the presence of one or more cardiorespiratory disease-VOC biomarkers in a sample of exhaled air from the subject,
wherein the presence of one or more of the VOC biomarkers in the sample suggests the subject has a cardiorespiratory disease, and
administering a therapeutic agent to the subject, in order to treat the cardiorespiratory disease.

3. (canceled)

4. A method of selecting a subject for treatment with a therapeutic agent or composition for a cardiorespiratory disease, the method comprising:
detecting the presence of one or more cardiorespiratory disease-VOC biomarkers in a sample of exhaled air from the subject,
wherein the presence of one or more of the VOC biomarkers in the sample suggests the subject has a cardiorespiratory disease, and
selecting the subject for treatment with a therapeutic agent or composition for the cardiorespiratory disease.

5. The method of claim 2, further comprising:
determining if the therapeutic agent or composition is effectively treating thea cardiorespiratory disease in the subject, determining comprising:
determining the concentration of one or more cardiorespiratory disease-VOC biomarkers in a test sample that has been exhaled by the subject, and
comparing the concentration of the at least one or more VOCs in the test sample with the concentration in a reference sample,
wherein if the concentration of the one or more VOC biomarkers in the test sample is lower compared to the concentration in a reference sample, it is indicative that the therapeutic agent or composition is effectively treating the cardiorespiratory disease in the subject, and
further administering the therapeutic agent or composition according to the restuls of determining if the therapeutic agent or composition is effectively treating the cardiorespiratory disease.

6. The method according to claim 5, wherein the concentration of the VOC biomarker in the test sample is lower by (or reduced by at) least about 5%, 10%, 15%, 20%, 25%, 30%, 35%, 40%, 45%, 50%, 55%, 60%, 65%, 70%, 75%, 80%, 85%, 90%, 95% or 100% compared to the concentration in the reference sample.

7. The method according to claim 2, wherein the subject is experiencing breathlessness.

8. The method according to claim 2, wherein a two-dimensional gas chromatography coupled with mass spectrometry is used to detect the presence of the one or more VOC biomarkers in the sample.

9. The method according to claim 2, wherein the cardiorespiratory disease is one or more diseases selected from the group comprising: asthma, COPD, heart failure and pneumonia.

10. (canceled)

11. The method according to claim 2, wherein the one or more cardiorespiratory disease-VOC biomarkers is a selection of one or more of the following: hexane; octane; tetradecane,2,3-butanedione; hexanal; 2-methyl-2-propenal; 1-hexadecanol; 2-methyl-1,3-dioxolane; limonene; eucalyptol; menthone; p-mentha-1,4/8-diene; 3-carene; beta phellandrene; sesquiterpenoid; xylene; 2,3-dimethylnapthalene; carbonyl sulphide; 4-cyanocylohexene; methenamine; dichloromethane; N,N-dimethyl-1-nonanamine; and a alkenyl hexanoic acid ester.

12. (canceled)

13. The method according to claim 11, wherein the one or more biomarkers is one or more asthma-VOC biomarkers and is a selection of one or more of the following: 3-methylpentane; 2-methylnonane; decane; 1-nonene; methyldecanal isomer; undecanal; 3-methylbenzaldehyde; 2-ethylhexanol; tetrahydrofuran; 1,4-dioxane; beta-bisabolene; and N,N-dimethyl-1-dodecanamine.

14. The method according to claim 11, wherein the one or more biomarkers is one or more COPD-VOC biomarkers and is a selection of one or more of the following: nonane; 4-methylundecane; 1-decanol; menthol; camphene; galaxolide; 3-methyl thiophene; and N,N-dimethyl-1-dodecanamine.

15. The method according to claim 11, wherein the one or more biomarkers is one or more heart failure-VOC biomarkers and is a selection of one or more of the following: undecane; cyclohexene; butanal; 2-methyl-2-propenal; tridecanal; ethyl acetate;
1,3-dioxolane; beta myrcene; ethylbenzene; and decyl isobutyl ether.

16. The method according to claim 11, wherein the one or more biomarkers is one or more pneumonia-VOC biomarkers and is a selection of one or more of the following: 2,6-dimethyloctane; diemthylundecane isomer; 1-decene; 3-buten-2-one (methyl vinyl ketone); 1-(methylthio)-1-propene; 1-methylthio-propane; and dodecylacryalte.

17. The method according to claim 4, wherein the subject is experiencing breathlessness.

18. The method according to claim 4, wherein a two-dimensional gas chromatography coupled with mass spectrometry is used to detect the presence of the one or more VOC biomarkers in the sample.

19. The method according to claim 4, wherein the one or more cardiorespiratory disease-VOC biomarkers is a selection of one or more of the following: hexane; octane; tetradecane,2,3-butanedione; hexanal; 2-methyl-2-propenal; 1-hexadecanol; 2-methyl-1,3-dioxolane; limonene; eucalyptol; menthone; p-mentha-1,4/8-diene; 3-carene; beta phellandrene; sesquiterpenoid; xylene; 2,3-dimethylnapthalene; carbonyl sulphide; 4-cyanocylohexene; methenamine; dichloromethane; N,N-dimethyl-1-nonanamine; and a alkenyl hexanoic acid ester.

20. The method according to claim 19, wherein the one or more biomarkers is one or more asthma-VOC biomarkers and is a selection of one or more of the following: 3-methylpentane; 2-methylnonane; decane; 1-nonene; methyldecanal isomer; undecanal; 3-methylbenzaldehyde; 2-ethylhexanol; tetrahydrofuran; 1,4-dioxane; beta-bisabolene; and N,N-dimethyl-1-dodecanamine.

21. The method according to claim 19, wherein the one or more biomarkers is one or more COPD-VOC biomarkers and is a selection of one or more of the following: nonane; 4-methylundecane; 1-decanol; menthol; camphene; galaxolide; 3-methyl thiophene; and N,N-dimethyl-1-dodecanamine.

22. The method according to claim 19, wherein the one or more biomarkers is one or more heart failure-VOC biomarkers and is a selection of one or more of the following: undecane; cyclohexene; butanal; 2-methyl-2-propenal; tridecanal; ethyl acetate; 1,3-dioxolane; beta myrcene; ethylbenzene; and decyl isobutyl ether.

23. The method according to claim 19, wherein the one or more biomarkers is one or more pneumonia-VOC biomarkers and is a selection of one or more of the following: 2,6-dimethyloctane; diemthylundecane isomer, 1-decene; 3-buten-2-one (methyl vinyl ketone); 1-(methylthio)-1-propene; 1-methylthio-propane; and dodecylacryalte.
